(19) United States
(12) Patent Application Publication
(10) Pub. No.: US 2017/0327508 A1
(43) Pub. Date: Nov. 16, 2017

(54) DEUTERATED HETEROARYL SULFONAMIDES AND THEIR USE

(71) Applicant: ChemoCentryx, Inc., Mountain View, CA (US)

(72) Inventors: Penglie Zhang, Foster City, CA (US); Shichang Miao, Foster City, CA (US)

(21) Appl. No.: 15/596,278

(22) Filed: May 16, 2017

Related U.S. Application Data
(60) Provisional application No. 62/337,094, filed on May 16, 2016.

(51) Int. Cl. C07D 487/04; C07B 59/00; C07D 471/04

(52) U.S. Cl. CPC C07D 487/04; C07D 471/04; C07B 59/002; C07B 2200/05

(57) ABSTRACT

Compunds are provided that act as potent antagonists of the CCR2 receptor. These compounds are useful for treating inflammation, a hallmark disease for CCR2. The compounds are generally aryl sulfonamide derivatives that include deuterium modifications and are useful in pharmaceutical compositions, methods for the treatment of CCR2-mediated diseases, and as controls in assays for the identification of CCR2 antagonists.

This application claims the benefit under 35 U.S.C. §119(e) of U.S. Provisional Patent Application No. 62/337,094, filed May 16, 2016, and titled “DEUTERATED HETEROARYL SULFONAMIDES AND THEIR USE,” which is incorporated, in its entirety, by this reference.

### FIELD

The present disclosure describes novel compounds and compositions effective in inhibiting the binding or function of various chemokines to chemokine receptors. The compounds include deuterium modification to improve the metabolic properties of the drug. Also disclosed herein are methods for treating a CCR2-mediated condition or disease comprising administration of the compounds and composition described herein.

### BACKGROUND

The present disclosure provides compounds, pharmaceutical compositions containing one or more of those compounds or their pharmaceutically acceptable salts, which are effective in inhibiting the binding or function of various chemokines to chemokine receptors. As antagonists or modulators of chemokine receptors, the compounds and compositions have utility in treating various immune disorders, conditions, and diseases including immune realted disorders.

Chemokines, also known as chemotactic cytokines, are a group of small molecular-weight proteins that are released by a wide variety of cells and have a variety of biological activities. Chemokines attract various types of cells of the immune system, such as macrophages, T cells, eosinophils, basophils and neutrophils, and cause them to migrate from the blood to various lymphoid and none-lymphoid tissues. They mediate infiltration of inflammatory cells to sites of inflammation, and are responsible for the initiation and perpetuation of many inflammation diseases (reviewed in Schall, Cytokine, 3:165-183 (1991), Schall et al., Curr. Opin. Immunol., 6:865-873 (1994)).

In addition to stimulating chemotaxis, chemokines can induce other changes in responsive cells, including changes in cell shape, granule exocytosis, integrin up-regulation, formation of bioactive lipids (e.g., leukotrienes), respiratory burst associated with leukocyte activation, cell proliferation, resistance to induction of apoptosis and angiogenesis. Thus, chemokines are early triggers of the inflammatory response, causing inflammatory mediator release, chemotaxis and extravasation to sites of infection or inflammation. They are also stimulators of a multitude of cellular processes that bear important physiological functions as well as pathological consequences.

Chemokines exert their effects by activating chemokine receptors expressed by responsive cells. Chemokine receptors are a class of G-protein coupled receptors, also known as seven-transmembrane receptors, found on the surface of a wide variety of cell types such as leukocytes, endothelial cells, smooth muscle cells and tumor cells.

Chemokines and chemokine receptors are expressed by intrinsic renal cells and infiltrating cells during renal inflammation (Segerer et al., J. Am. Soc. Nephrol., 11:152-76 (2000); Morii et al., J. Diabetes Complications, 17:11-5 (2003); Lloyd et al. J. Exp. Med., 185:1371-80 (1997); Gonzalez-Cuadrado et al. Clin. Exp. Immunol, 106:518-22 (1996); Eddy & Giachelli, Kidney Int., 47:1546-57 (1995); Diamond et al., Am. J. Physiol., 266:F926-33 (1994)). In humans, CCR2 and ligand MCP-1 are among the proteins expressed in renal fibrosis, and are correlated with the extent of macrophage infiltration into the interstitium (Yang et al., Zhonghua Yi Xue Za Zhi, 81:73-7 (2001); Stephan et al., J. Urol., 167:1497-502 (2002); Amann et al., Diabetes Care, 26:2421-5 (2003); Dai et al., Chin. Med. J. (Engl), 114:864-8 (2001)). In animal models of renal fibrosis, blockade of CCR2 or MCP-1 leads to a marked reduction in severity of renal inflammation (Kitagawa et al., Am. J. Pathol., 165:237-46 (2004); Wada et al., Am. J. Pathol., 165:237-46 (2004); Shimizu et al., J. Am. Soc. Nephrol., 14:1496-505 (2003)).

Rheumatoid arthritis is a chronic disease of the joints characterized by synovial inflammation that leads to the destruction of cartilage and bone. Although the underlying causes of the disease are unknown, it is believed that macrophages and Th-1 type T cells play a key role in the initiation and perpetuation of the chronic inflammatory process (Vervoordeldonk et al., Curr. Rheumatol. Rep., 4:208-17 (2002)).

MCP-1 is among the several chemokines, including MIP-1α and IL-8, identified in rheumatoid synovium (Villiger et al., J. Immunol., 149:722-7 (1992); Scaife et al., Rheumatology (Oxford), 43:1346-52 (2004); Shadidi et al., Scand. J. Immunol., 57:192-8 (2003); Taylor et al., Arthritis Rheum., 43:38-47 (2000); Tucci et al., Biomed. Sci. Instrum., 34:169-74 (1997)). Chemokine receptors CCR1, CCR2, CCR3 and CCR5 are up-regulated in the joints from arthritic mice (Plater-Zyberk et al., Immunol. Lett., 57:117-20 (1997). Blockade of MCP-1 activity using a CCR2 antagonist or an antibody against MCP-1 have been shown efficacious in reducing joint inflammation in experimental models of rheumatoid arthritis (Gong et al., J. Exp. Med., 186:131-7 (1997); Ogata et al., J. Pathol., 182:106-14 (1997)).

Chemokine receptor-mediated infiltration of macrophages in the fat tissues may also contribute to the complications arising from obesity, a condition resulting from excessive storage of fat in the body. Obesity predisposes the affected individuals to many disorders, such as non-insulin-dependent diabetes, hypertension, stroke, and coronary artery disease. In obesity, adipose tissues have altered metabolic and endocrine functions that lead to an increased release of fatty acids, hormones, and pro-inflammatory molecules. Adipose tissue macrophages are believed to be a key source of pro-inflammatory cytokines including TNF-alpha, iNOS and IL-6 (Weisberg et al., J. Clin. Invest., 112:1796-808 (2003)). Recruitment of macrophages to the adipose tissue is likely mediated by MCP-1 produced by adipocytes (Christiansen T, et al., Int J Obes (Lond). 2005 January;29(1):146-50; Sartipy et al., Proc. Natl. Acad. Sci. U.S.A., 100:7265-70 (2003)).

Elevated MCP-1 may induce adipocyte differentiation and insulin resistance, and contribute to pathologies associated with hyper-insulinemia and obesity. MCP-1 is over-expressed in plasma in obese mice compared to lean controls and white adipose is a major source. MCP-1 has also been shown to accelerate wound healing, and has a direct angiogenic effect on epithelial cells, and may play a direct role in the remodeling of adipose tissue in obesity. (Sartipy P, Loskutoff D J., Proc. Natl. Acad. Sci. U.S.A., 100:7265 (2003)).

MCP-1 plasma levels are substantially increased in Diet Induce Obesity (DIO) mice, and a strong correlation between plasma MCP-1 levels and body weight has been identified. Furthermore, elevation of MCP-1 induced by high fat diet causes changes in the CD11b positive monocyte population in DIO mice. (Takahashi K, et al., J. Biol. Chem., 46654 (2003)).

Furthermore, chronic inflammation in fat is thought to play a crucial role in the development of obesity-related insulin resistance (Xu H, et al., J Clin Invest. 2003 December; 112(12):1821-30). It has been proposed that obesity related insulin resistance is, at least in part, a chronic inflammatory disease initiated in adipose tissue. Many inflammation and macrophage specific genes are dramatically upregulated in white adipose tissue in mouse models of genetic and high fat diet-induced obesity (DIO), and this upregulation precedes a dramatic increase in circulating insulin.

Increased expression levels of monocyte CCR2 and monocyte chemoattractant protein-1 in patients with diabetes mellitus (Biochemical and Biophysical Research Communications, 344(3):780-5 (2006)) were found in a study involving diabetic patients. Serum MCP-1 concentrations and surface expression of CCR2 on monocytes in diabetic patients were significantly higher than in non-diabetics, and the serum MCP-1 levels correlated with HbA1c, triglycerides, BMI, hs-CRP. Surface expression levels of CD36 and CD68 on monocytes were significantly increased in diabetic patients and more unregulated by MCP-1 in diabetics, augmenting uptake of ox-LDL, and hence potentially foam cell transformation. Elevated serum MCP-1 and increased monocyte CCR2, CD36, CD68 expression correlated with poor blood glucose control and potentially correlate with increased vessel wall monocyte recruitment.

MCP-1 is a potential player in negative cross talk between adipose tissue and skeletal muscle (Bianco J J, et al., Endocrinology, 2458 (2006)). MCP-1 can significantly reduce insulin-stimulated glucose uptake, and is a prominent inducer of insulin resistance in human skeletal muscle cell. Adipose tissue is a major secretory and endocrine active organ producing bioactive proteins regulating energy metabolism and insulin sensitivity.

CCR2 modulates inflammatory and metabolic effects of high-fat feeding (Weisberg S P, et al., J. Clin. Invest., 115 (2006)). Genetic deficiency in CCR2 reduced food intake and attenuated the development of obesity in mice fed a high fat diet. In obese mice matched for adiposity, CCR2 deficiency reduced macrophage content and inflammatory profile of adipose tissue, increased adiponectin expression, and improved glucose homeostatis and insulin sensitivity. In lean animals, no effect of CCR2 genotype on metabolic trait was found. In high-fat diet mice, CCR2 genotype modulated feeding, the development of obesity and adipose tissue inflammation. Once established, short term antagonism was shown to attenuate macrophage accumulation in adipose tissue and insulin resistance.

Chemokine and chemokine receptors are the key regulators of immune cell trafficking. MCP-1 is a potent chemoattractant of monocytes and T cells; its expression is induced under inflammatory conditions including proinflammatory cytokine stimulations and hypoxia. The interaction between MCP-1 and CCR2 mediates migration of monocytes, macrophage as well as activated T cells and play a key role in the pathogenesis of many inflammatory diseases. Inhibition of CCR2 functions using small molecule antagonists described in this disclosure represents a new approach for the treatments of inflammatory disorders.

Psoriasis is a chronic inflammatory disease characterized by hyperproliferation of keratinocytes and pronounced leukocyte infiltration. It is known that keratinocytes from psoriasis lesion express abundant CCR2 ligand MCP-1, particularly when stimulated by proinflammatory cytokines such as TNF-α (Vestergaard et al., Acta. Derm. Venereol., 84(5):353-8 (2004); Gillitzer et al., J. Invest. Dermatol., 101(2):127-31 (1993); Deleuran et al., J. Dermatol. Sci., 13(3):228-36 (1996)). Since MCP-1 can attract migration of both macrophages and dendritic cells expressing CCR2 to the skin, this receptor and ligand pair is believed to be important in regulating the interaction between proliferating keratinocytes and dermal macrophage during the development of psoriasis. A small molecule antagonist may thus be useful in the treatment of psoriasis.

In addition to inflammatory diseases, chemokines and chemokine receptors have also been implicated in cancers (Broek et al., Br. J. Cancer, 88(6):855-62 (2003)). Tumor cells stimulate the formation of stroma that secretes various mediators pivotal for tumor growth, including growth factors, cytokines, and proteases. It is known that the level of MCP-1 is associated significantly with tumor-associated macrophage accumulation, and prognostic analysis reveals that high expression of MCP-1 is a significant indicator of early relapse in breast cancer (Ueno et al., Clin. Cancer Res., 6(8):3282-9 (2001)). A small molecule antagonist of a chemokine may thus be able to reduce the release of growth-stimulating cytokines by blocking accumulation of macrophages at sites of tumor formation.

A potentially attractive strategy for improving a drug's metabolic properties is deuterium modification. Deuterium is a safe, stable, non-radioactive isotope of hydrogen. Compared to hydrogen, deuterium forms stronger bonds with carbon. The increased bond strength imparted by a deuterium can positively impact the ADME properties of a drug, creating the potential for improved drug efficacy, safety, and/or tolerability. Drugs have multiple sites where metabolism is possible. Replacing one or more hydrogen by one or more deuterium at one or more of these site(s) may affect the metabolism of the drug. Deuterium can also have a secondary effect which may modify the properties of the drug creating the potential for improved properties such as drug efficacy, safety, and/or tolerability.

### BRIEF SUMMARY

Thus, the present disclosure is directed to deuterated compounds and pharmaceutically acceptable salts thereof, pharmaceutical compositions, and methods useful in modulating chemokine activity. The compounds and salts thereof, compositions, and methods described herein are useful in treating or preventing chemokine-mediated conditions or diseases, including certain inflammatory and immunoregulatory disorders and diseases.

The compounds of the present disclosure are believed to modulate one or more of CCR2, CCR3, CCR4, CCR5, CCR6, CCR7, CCR8, CCR10, CXCR3, CXCR4, CXCR5, and CX3CR1. In particular, various compounds of the present disclosure modulate CCR2.

In one embodiment, the compounds of the present disclosure are represented by formula (I), or salts thereof:

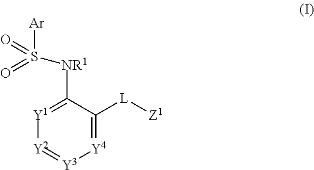

where Ar is selected from the group consisting of substituted or unsubstituted optionally deuterated C₆₋₁₀ aryl and substituted or unsubstituted optionally deuterated 5- to 10-membered heteroaryl.

R¹ is selected from the group consisting of hydrogen or deuterium, substituted or unsubstituted C₁₋₈ alkyl, substituted or unsubstituted optionally deuterated C₂₋₆ alkenyl, substituted or unsubstituted optionally deuterated C₂₋₆ alkynyl, and substituted or unsubstituted optionally deuterated 3- to 10-membered heterocyclyl;

Y¹ is selected from the group consisting of —CR^{2a}—, —N—, and —N⁺(O)⁻—;

Y² is selected from the group consisting of —CR^{2b}—, —N—, and —N⁺(O)⁻—;

Y³ is selected from the group consisting of —CR^{2c}—, —N—, and —N⁺(O)⁻—;

R^{2a}, R^{2b} and R^{2c} are each independently selected from the group consisting of hydrogen or deuterium, halogen, —CN, —C(O)R³, —CO₂R³, —C(O)NR³R⁴, —OR³, —OC(O)R³, —OC(O)NR³R⁴, —SR³, —S(O)R³, —S(O)₂R³, —S(O)₂NR³R⁴, —NO₂, —NR³R⁴, —NR³C(O)R⁴, —NR³C(O)OR⁴, —NR³S(O)₂R⁴, —NR³C(O)NR⁴R⁵, substituted or unsubstituted optionally deuterated C₁₋₈ alkyl, substituted or unsubstituted optionally deuterated C₂₋₈ alkenyl, substituted or unsubstituted optionally deuterated C₂₋₈ alkynyl, substituted or unsubstituted optionally deuterated 3- to 10-membered heterocyclyl, substituted or unsubstituted optionally deuterated C₆₋₁₀ aryl, and substituted or unsubstituted optionally deuterated 5- to 10-membered heteroaryl;

R³, R⁴, and R⁵ are each independently selected from the group consisting of hydrogen or deuterium, substituted or unsubstituted optionally deuterated C₁₋₈ alkyl, substituted or unsubstituted optionally deuterated C₂₋₈ alkenyl, substituted or unsubstituted optionally deuterated C₂₋₈ alkynyl, substituted or unsubstituted optionally deuterated C₆₋₁₀ aryl, substituted or unsubstituted optionally deuterated 5- to 10-membered heteroaryl, and substituted or unsubstituted optionally deuterated 3- to 10-membered heterocyclyl;

R³ and R⁴, R⁴ and R⁵ or R³ and R⁵ may, together with the atoms to which they are attached, form a substituted or unsubstituted optionally deuterated 5-, 6-, or 7-membered ring;

L is selected from the group consisting of a bond, —O—, —S—, —S(O)—, —S(O)₂—, —CR⁶R⁷—, —NR⁸—, —C(O)—, —C(═N—O—R⁹)—, —C(O)NR⁸—, and —NR⁸C(O)—;

R⁶ and R⁷ are each independently selected from the group consisting of hydrogen or deuterium, halogen, substituted or unsubstituted optionally deuterated C₁₋₈ alkyl, substituted or unsubstituted optionally deuterated 3- to 10-membered heterocyclyl, substituted or unsubstituted optionally deuterated C₂₋₆ alkenyl, substituted or unsubstituted optionally deuterated C₂₋₆ alkynyl, —CN, —OR⁹, —NR¹⁰R¹¹, —S(O)R⁹, and —S(O)₂R⁹;

R⁶ and R⁷ may, together with the carbon atom to which they are attached, form substituted or unsubstituted optionally deuterated C₃₋₈ cycloalkyl or substituted or unsubstituted optionally deuterated 3- to 10-membered heterocyclic ring;

R⁹ is selected from the group consisting of hydrogen or deuterium, substituted or unsubstituted optionally deuterated C₁₋₈ alkyl, substituted or unsubstituted optionally deuterated C₂₋₈ alkenyl, substituted or unsubstituted optionally deuterated C₂₋₈ alkynyl, substituted or unsubstituted optionally deuterated C₆₋₁₀ aryl, substituted or unsubstituted optionally deuterated 5- to 10-membered heteroaryl, and substituted or unsubstituted optionally deuterated 3- to 10-membered heterocyclyl;

R¹⁰ and R¹¹ are each independently selected from the group consisting of substituted or unsubstituted optionally deuterated C₁₋₈ alkyl, substituted or unsubstituted optionally deuterated 3- to 10-membered heterocyclyl, substituted or unsubstituted optionally deuterated C₆₋₁₀ aryl, substituted or unsubstituted optionally deuterated 5- to 10-membered heteroaryl, substituted or unsubstituted optionally deuterated C₂₋₈ alkenyl, and substituted or unsubstituted optionally deuterated C₂₋₈ alkynyl;

R¹⁰ and R¹¹ of —NR¹⁰R¹¹ may, together with the nitrogen, form substituted or unsubstituted optionally deuterated 3- to 10-membered heterocyclyl;

R⁸ is selected from the group consisting of hydrogen or deuterium, —C(O)R¹², —S(O)₂R¹², —CO₂R¹², substituted or unsubstituted optionally deuterated C₁₋₈ alkyl, substituted or unsubstituted optionally deuterated 3- to 10-membered heterocyclyl, substituted or unsubstituted optionally deuterated C₂₋₆ alkenyl, and substituted or unsubstituted optionally deuterated C₂₋₆ alkynyl;

R¹² is selected from the group consisting of substituted or unsubstituted optionally deuterated C₁₋₈ alkyl, substituted or unsubstituted optionally deuterated C₂₋₆ alkenyl, substituted or unsubstituted optionally deuterated C₂₋₆ alkynyl, substituted or unsubstituted optionally deuterated 3- to 10-membered heterocyclyl, substituted or unsubstituted optionally deuterated C₆₋₁₀ aryl, and substituted or unsubstituted optionally deuterated 5- to 10-membered heteroaryl;

Z¹ is selected from the group consisting of substituted or unsubstituted optionally deuterated C₆₋₁₀ aryl, substituted or unsubstituted optionally deuterated 5- to 10-membered heteroaryl, substituted or unsubstituted optionally deuterated 3- to 10-membered heterocyclyl, and —NR¹³R¹⁴;

R¹³ and R¹⁴ are each independently selected from the group consisting of hydrogen or deuterium, substituted or unsubstituted optionally deuterated C₁₋₈ alkyl, substituted or unsubstituted C₂₋₈ alkenyl, substituted or unsubstituted optionally deuterated C₂₋₈ alkynyl, substituted or unsubstituted optionally deuterated 3- to 10-membered heterocyclyl, substituted or unsubstituted optionally deuterated C₆₋₁₀ aryl, substituted or unsubstituted optionally deuterated 5- to 10-membered heteroaryl, substituted or unsubstituted optionally deuterated (C₁₋₄ alkyl)-(C₆₋₁₀ aryl), and substituted or unsubstituted optionally deuterated (C₁₋₄ alkyl)- (5- to 10-membered heteroaryl);

R¹³ and R¹⁴ may, together with the nitrogen, form a substituted or unsubstituted optionally deuterated 4-, 5-, 6-, or 7-membered heterocyclyl.

Y⁴ is selected from the group consisting of —N— and —N⁺(O)⁻—;

Wherein the compound comprises at least one deuterium above natural isotopic abundance, or a pharmaceutically acceptable salt thereof.

In one aspect of the embodiment, the compound as described herein wherein at least one of Y¹, Y², Y³ or Z¹ comprises deuterium above natural isotopic abundance.

In one embodiment, the compounds of the present disclosure are represented by formula (II), or salts thereof:

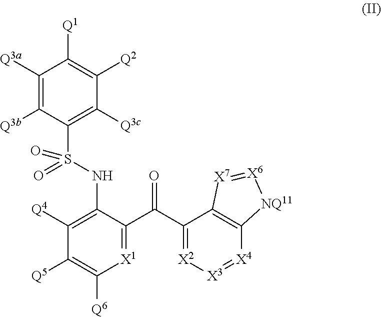

each of Q¹ and Q² independently is hydrogen, deuterium, halogen, optionally deuterated c₁₋₈ alkyl, —CN, or optionally deuterated C₁₋₈ haloalkyl;

each Q^{3b}, Q^{3b}, Q^{3c} independently is hydrogen or deuterium;

Q⁴ is hydrogen or deuterium;

Q⁵ is halogen or optionally deuterated C₁₋₈ alkyl;

Q⁶ is hydrogen or deuterium;

each of X¹, X², X⁴, X⁶, and X⁷ independently is —CQ⁷—, —N—, or —NO—;

X³ is —CQ⁷—;

each Q⁷ independently is hydrogen or deuterium, halogen, substituted or unsubstituted optionally deuterated C₁₋₈ alkyl, substituted or unsubstituted optionally deuterated C₂₋₈ alkenyl, substituted or unsubstituted optionally deuterated C₂₋₈ alkynyl, —CN, ═O, —NO₂, —OQ⁸, —OC(O)Q⁸, —CO₂Q⁸,—C(O)Q⁸, —C(O)NQ⁹Q⁸, —OC(O)NQ⁹Q⁸, —NQ¹⁰C(O)Q⁸, —NQ¹⁰C(O)NQ⁹Q⁸, —NQ⁹Q⁸, —NQ¹⁰CO₂Q⁸, —SQ⁸, —S(O)Q⁸, —S(O)₂Q⁸, —S(O)₂NQ⁹Q⁸, —NQ¹⁰S(O)₂Q⁸, substituted or unsubstituted optionally deuterated C₆₋₁₀ aryl, substituted or unsubstituted optionally deuterated 5- to 10-membered heteroaryl and substituted or unsubstituted optionally deuterated 3- to 10-membered heterocyclyl;

each occurrence of Q⁸, Q⁹, and Q¹⁰ is independently selected from the group consisting of hydrogen or deuterium, optionally deuterated C₁₋₈ alkyl, optionally deuterated C₂₋₈ alkenyl, optionally deuterated C₂₋₈ alkynyl, optionally deuterated aryl, or optionally deuterated heteroaryl; or Q⁹ and Q⁸ or Q¹⁰ and Q⁸, together with the atom(s) to which they are attached, form an substituted or unsubstituted optionally deuterated 5-, 6-, or 7-membered ring;

Q¹¹ is selected from the group consisting of hydrogen, optionally deuterated C₁₋₈ alkyl, optionally deuterated C₂₋₈ alkenyl, optionally deuterated C₂₋₈ alkynyl, optionally deuterated substituted or unsubstituted C₆₋₁₀ aryl, optionally deuterated substituted or unsubstituted 5-to 10-membered heteroaryl, and optionally deuterated substituted or unsubstituted 3- to 10-membered heterocycle; and where in the compound comprises at least one deuterium above natural isotopic abundance, or a pharmaceutically acceptable salt thereof.

In one embodiment for the compound of Formula II as described herein, at least one of Q¹ or Q² is other than hydrogen. In one embodiment, each Q^{a3}, Q^{3b} and Q^{3c} independently is hydrogen or deuterium. In one embodiment, one or more Q⁴, Q⁵, Q⁶, or Q⁷ comprises deuterium above natural isotopic abundance. In one embodiment, Q¹ is halogen. In one embodiment, Q² is deuterated C₁₋₈ haloalkyl. In one embodiment, Q² is deuterated C₁₋₈ haloalkyl. In one embodiment, Q² is C₁₋₈ haloalkyl. In one embodiment, Q⁴ is deuterium. In one embodiment, Q⁴ is hydrogen. In one embodiment, Q⁵ is deuterated C₁₋₈ alky. In one embodiment, Q⁵ is C₁₋₈ alkyl. In one embodiment, Q⁶ is deuterium. In one embodiment, Q⁶ is hydrogen. In one embodiment, Q⁷ is deuterium. In one embodiment, Q⁷ is hydrogen.

In one embodiment, the compounds of the present disclosure are represented by formula (II), or salts thereof:

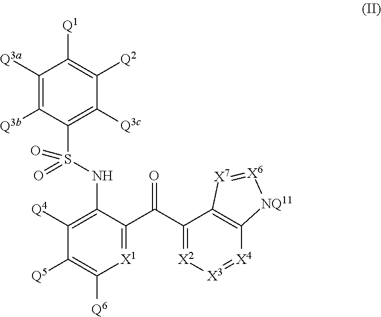

wherein each of Q¹ and Q² independently is hydrogen, deuterium, halogen, C₁₋₈ alkyl, —CN, or C₁₋₈ haloalkyl;

each Q^{3a}, Q^{3b}, Q^{3c} independently is deuterium or hydrogen;

Q⁴ is hydrogen or deuterium;

Q⁵ is halogen or optionally deuterated C₁₋₈ alkyl;

Q⁶ is hydrogen or deuterium;

each of X¹, X², X⁴, X⁶, and X⁷ independently is —CQ⁷—, —N—, or —NO—;

X³ is —CQ⁷—;

each Q⁷ independently is hydrogen or deuterium;

Q¹¹ is hydrogen; and

wherein the compound comprises at least one deuterium above natural isotopic abundance, or a pharmaceutically acceptable salt thereof.

In one embodiment, at least one of Q¹ or Q² is other than hydrogen. In one embodiment, one or more of Q⁴, Q⁵, Q⁶, or Q⁷ comprises deuterium above natural isotopic abundance.

In one embodiment the compounds of the present disclosure are compounds selected from the group consisting of:

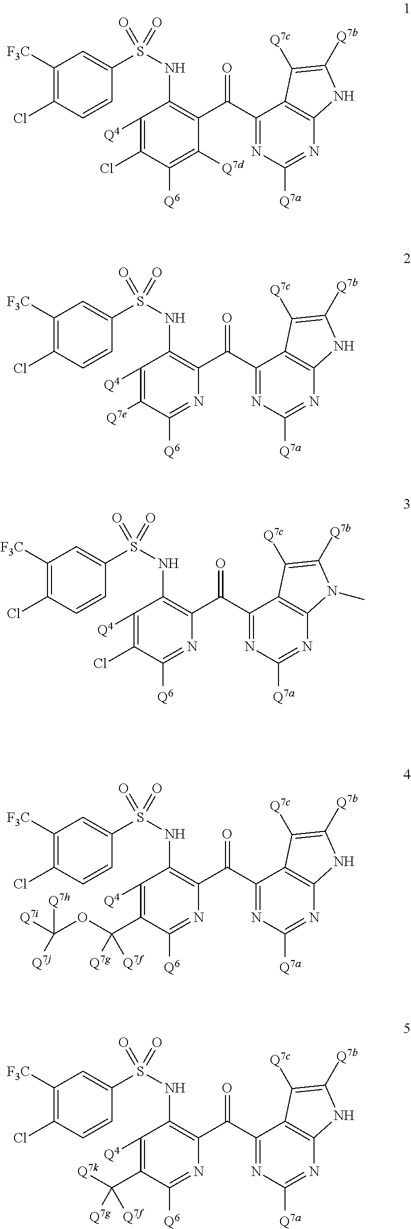

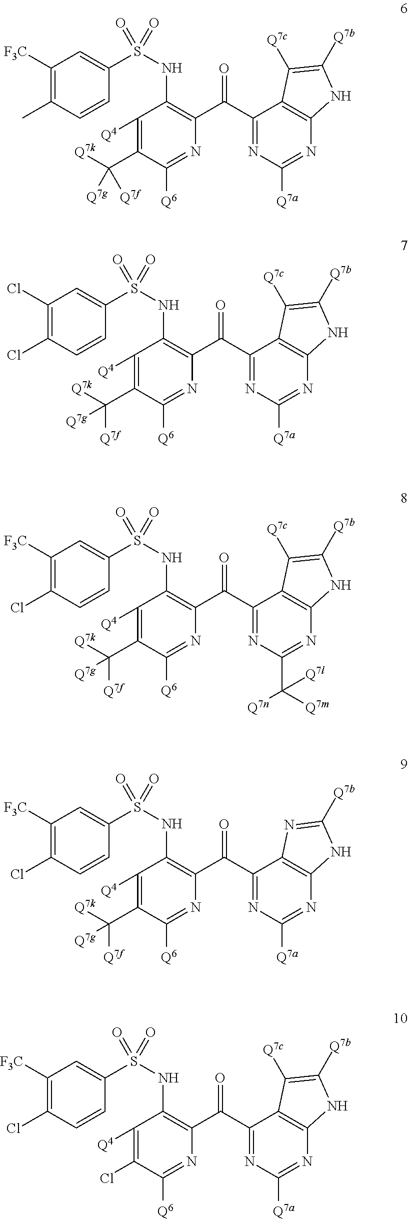

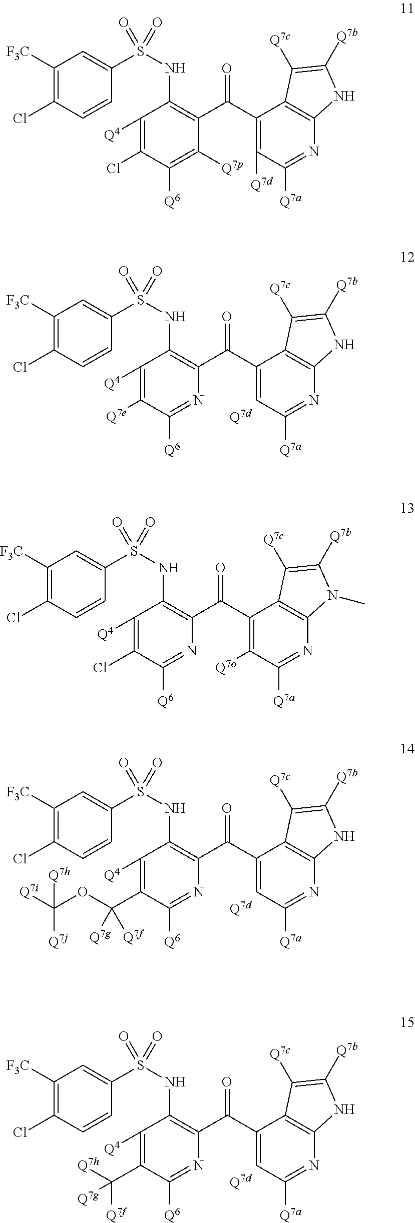

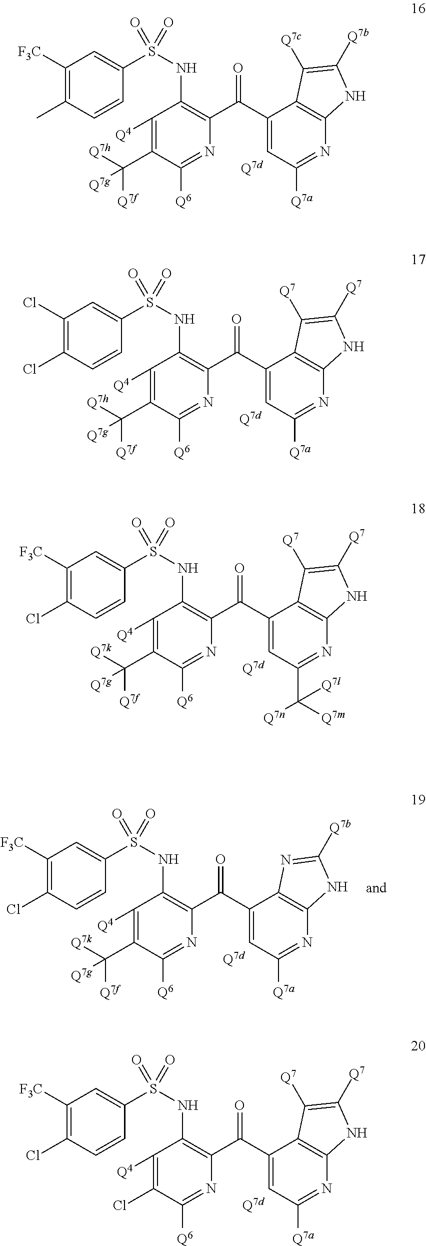

or a pharmaceutically acceptable salt thereof wherein each Q⁴ is independently hydrogen or deuterium;

each Q⁶ is independently hydrogen or deuterium;

each Q^{7a}, Q^{7b}, Q^{7c}, Q^{7d}, Q^{7e}, Q^{7f}, Q^{7g}, Q^{7h}, Q⁷ⁱ, Q^{7j}, Q^{7k}, Q^{7l}, Q^{7m}, Q⁷ⁿ, Q^{o} independently is hydrogen or deuterium; and wherein the compound comprises at least one deuterium above natural isotopic abundance.

In one embodiment, one or more of Q⁴, Q⁶, or Q^{7a-o} comprises deuterium above natural isotopic abundance. In one embodiment for each compound, or a pharmaceutically acceptable salt thereof, there is at least one deuterium above natural isotopic abundance. In one embodiment, there are two deuteriums above natural isotopic abundance. In one embodiment, there are three deuteriums above natural isotopic abundance. In one embodiment, each position represented as deuterium has deuterium enrichment of at least 50%. In one embodiment, each position represented as deuterium has deuterium enrichment of at least 90%.

In one embodiment, the compounds of the present disclosure are compounds selected from the group consisting of:

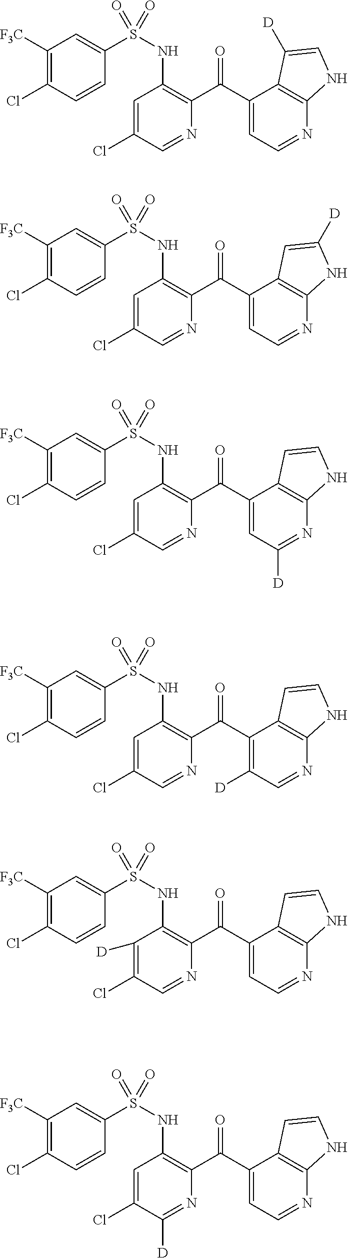 [SMILES: [2H]c1c[nH]c2nccc(C(=O)c3ncc(Cl)cc3NS(=O)(=O)c3ccc(Cl)c(C(F)(F)F)c3)c12 ; [2H]c1cc2c(C(=O)c3ncc(Cl)cc3NS(=O)(=O)c3ccc(Cl)c(C(F)(F)F)c3)ccnc2[nH]1 ; [2H]c1cc(C(=O)c2ncc(Cl)cc2NS(=O)(=O)c2ccc(Cl)c(C(F)(F)F)c2)c2cc[nH]c2n1 ; [2H]c1cnc2[nH]ccc2c1C(=O)c1ncc(Cl)cc1NS(=O)(=O)c1ccc(Cl)c(C(F)(F)F)c1 ; [2H]c1c(Cl)cnc(C(=O)c2ccnc3[nH]ccc23)c1NS(=O)(=O)c1ccc(Cl)c(C(F)(F)F)c1 ; [2H]c1nc(C(=O)c2ccnc3[nH]ccc23)c(NS(=O)(=O)c2ccc(Cl)c(C(F)(F)F)c2)cc1Cl]

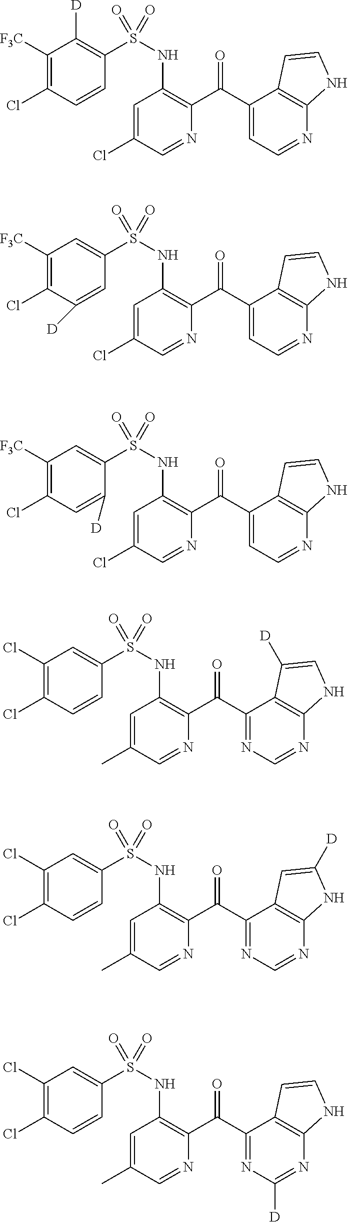 [SMILES: [2H]c1c(S(=O)(=O)Nc2cc(Cl)cnc2C(=O)c2ccnc3[nH]ccc23)ccc(Cl)c1C(F)(F)F ; [2H]c1cc(S(=O)(=O)Nc2cc(Cl)cnc2C(=O)c2ccnc3[nH]ccc23)cc(C(F)(F)F)c1Cl ; [2H]c1cc(Cl)c(C(F)(F)F)cc1S(=O)(=O)Nc1cc(Cl)cnc1C(=O)c1ccnc2[nH]ccc12 ; [2H]c1c[nH]c2ncnc(C(=O)c3ncc(C)cc3NS(=O)(=O)c3ccc(Cl)c(Cl)c3)c12 ; [2H]c1cc2c(C(=O)c3ncc(C)cc3NS(=O)(=O)c3ccc(Cl)c(Cl)c3)ncnc2[nH]1 ; [2H]c1nc(C(=O)c2ncc(C)cc2NS(=O)(=O)c2ccc(Cl)c(Cl)c2)c2cc[nH]c2n1]

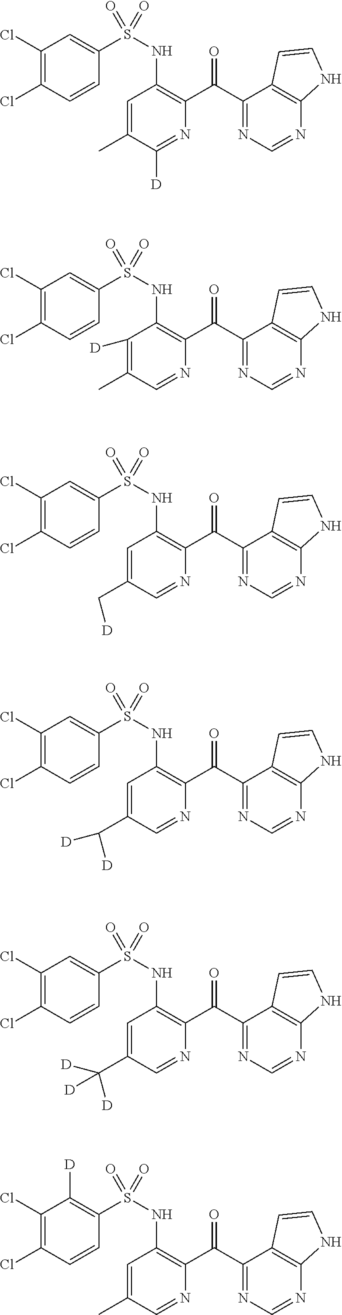 [SMILES: [2H]c1nc(C(=O)c2ncnc3[nH]ccc23)c(NS(=O)(=O)c2ccc(Cl)c(Cl)c2)cc1C ; [2H]c1c(C)cnc(C(=O)c2ncnc3[nH]ccc23)c1NS(=O)(=O)c1ccc(Cl)c(Cl)c1 ; [2H]Cc1cnc(C(=O)c2ncnc3[nH]ccc23)c(NS(=O)(=O)c2ccc(Cl)c(Cl)c2)c1 ; [2H]C([2H])c1cnc(C(=O)c2ncnc3[nH]ccc23)c(NS(=O)(=O)c2ccc(Cl)c(Cl)c2)c1 ; [2H]C([2H])([2H])c1cnc(C(=O)c2ncnc3[nH]ccc23)c(NS(=O)(=O)c2ccc(Cl)c(Cl)c2)c1 ; [2H]c1c(S(=O)(=O)Nc2cc(C)cnc2C(=O)c2ncnc3[nH]ccc23)ccc(Cl)c1Cl]

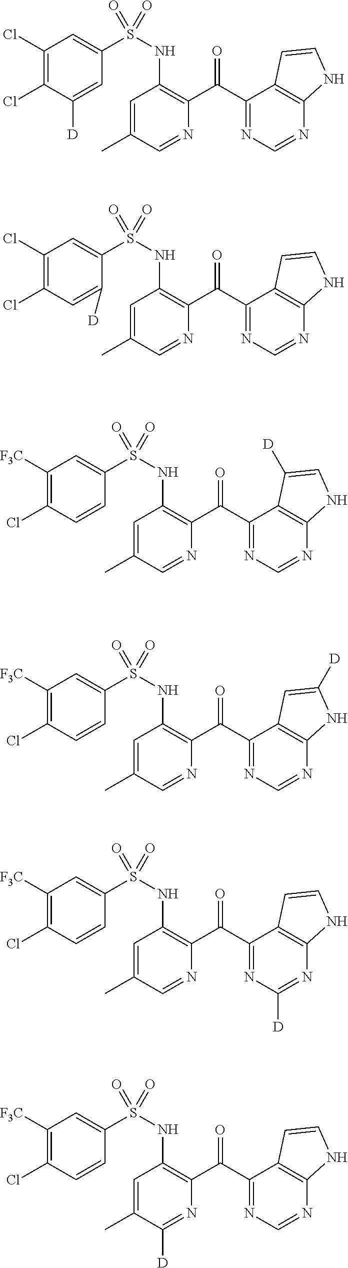 [SMILES: [2H]c1cc(S(=O)(=O)Nc2cc(C)cnc2C(=O)c2ncnc3[nH]ccc23)cc(Cl)c1Cl ; [2H]c1cc(Cl)c(Cl)cc1S(=O)(=O)Nc1cc(C)cnc1C(=O)c1ncnc2[nH]ccc12 ; [2H]c1c[nH]c2ncnc(C(=O)c3ncc(C)cc3NS(=O)(=O)c3ccc(Cl)c(C(F)(F)F)c3)c12 ; [2H]c1cc2c(C(=O)c3ncc(C)cc3NS(=O)(=O)c3ccc(Cl)c(C(F)(F)F)c3)ncnc2[nH]1 ; [2H]c1nc(C(=O)c2ncc(C)cc2NS(=O)(=O)c2ccc(Cl)c(C(F)(F)F)c2)c2cc[nH]c2n1 ; [2H]c1nc(C(=O)c2ncnc3[nH]ccc23)c(NS(=O)(=O)c2ccc(Cl)c(C(F)(F)F)c2)cc1C]

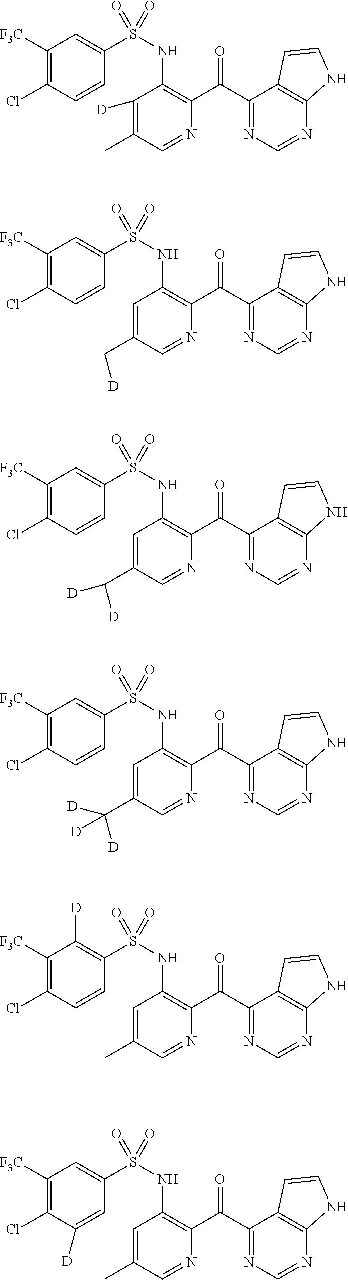 [SMILES: [2H]c1c(C)cnc(C(=O)c2ncnc3[nH]ccc23)c1NS(=O)(=O)c1ccc(Cl)c(C(F)(F)F)c1 ; [2H]Cc1cnc(C(=O)c2ncnc3[nH]ccc23)c(NS(=O)(=O)c2ccc(Cl)c(C(F)(F)F)c2)c1 ; [2H]C([2H])c1cnc(C(=O)c2ncnc3[nH]ccc23)c(NS(=O)(=O)c2ccc(Cl)c(C(F)(F)F)c2)c1 ; [2H]C([2H])([2H])c1cnc(C(=O)c2ncnc3[nH]ccc23)c(NS(=O)(=O)c2ccc(Cl)c(C(F)(F)F)c2)c1 ; [2H]c1c(S(=O)(=O)Nc2cc(C)cnc2C(=O)c2ncnc3[nH]ccc23)ccc(Cl)c1C(F)(F)F ; [2H]c1cc(S(=O)(=O)Nc2cc(C)cnc2C(=O)c2ncnc3[nH]ccc23)cc(C(F)(F)F)c1Cl]

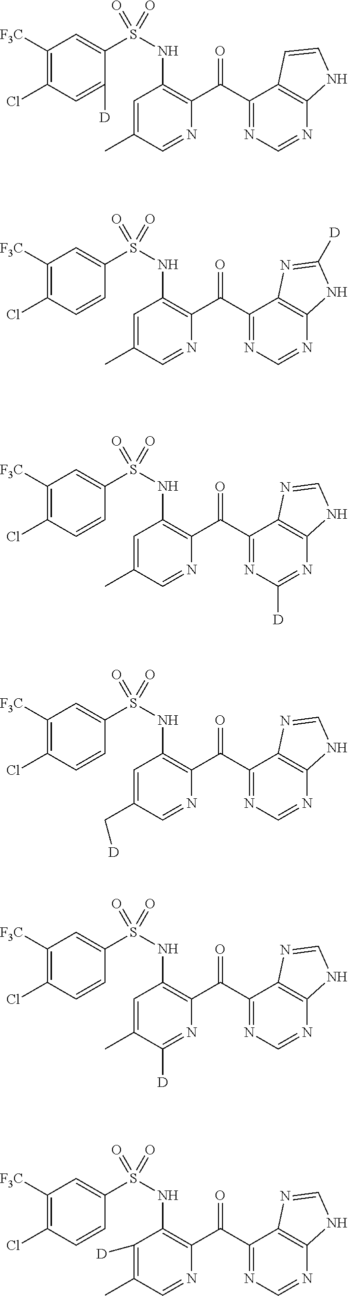 [SMILES: [2H]c1cc(Cl)c(C(F)(F)F)cc1S(=O)(=O)Nc1cc(C)cnc1C(=O)c1ncnc2[nH]ccc12 ; [2H]c1nc2c(C(=O)c3ncc(C)cc3NS(=O)(=O)c3ccc(Cl)c(C(F)(F)F)c3)ncnc2[nH]1 ; [2H]c1nc(C(=O)c2ncc(C)cc2NS(=O)(=O)c2ccc(Cl)c(C(F)(F)F)c2)c2nc[nH]c2n1 ; [2H]Cc1cnc(C(=O)c2ncnc3[nH]cnc23)c(NS(=O)(=O)c2ccc(Cl)c(C(F)(F)F)c2)c1 ; [2H]c1nc(C(=O)c2ncnc3[nH]cnc23)c(NS(=O)(=O)c2ccc(Cl)c(C(F)(F)F)c2)cc1C ; [2H]c1c(C)cnc(C(=O)c2ncnc3[nH]cnc23)c1NS(=O)(=O)c1ccc(Cl)c(C(F)(F)F)c1]

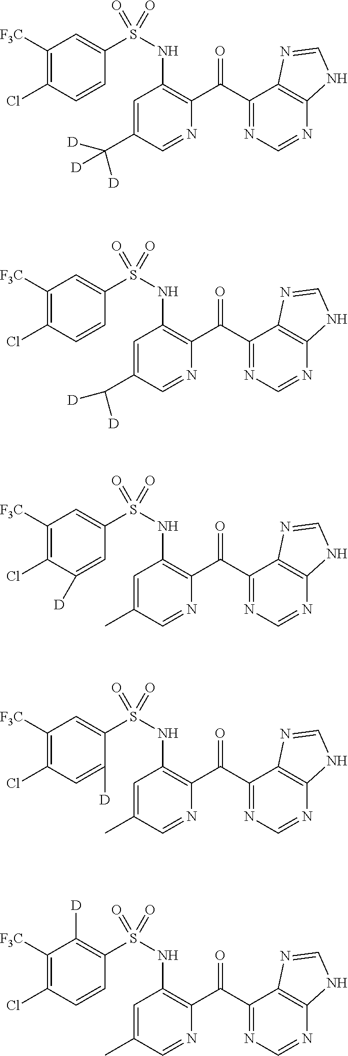 [SMILES: [2H]C([2H])([2H])c1cnc(C(=O)c2ncnc3[nH]cnc23)c(NS(=O)(=O)c2ccc(Cl)c(C(F)(F)F)c2)c1 ; [2H]C([2H])c1cnc(C(=O)c2ncnc3[nH]cnc23)c(NS(=O)(=O)c2ccc(Cl)c(C(F)(F)F)c2)c1 ; [2H]c1cc(S(=O)(=O)Nc2cc(C)cnc2C(=O)c2ncnc3[nH]cnc23)cc(C(F)(F)F)c1Cl ; [2H]c1cc(Cl)c(C(F)(F)F)cc1S(=O)(=O)Nc1cc(C)cnc1C(=O)c1ncnc2[nH]cnc12 ; [2H]c1c(S(=O)(=O)Nc2cc(C)cnc2C(=O)c2ncnc3[nH]cnc23)ccc(Cl)c1C(F)(F)F]

or a pharmaceutically acceptable salt thereof.

One embodiment of the disclosure includes a pharmaceutical composition comprising any of the compound described herein or a pharmaceutically acceptable salt thereof and a pharmaceutically acceptable carrier.

One embodiment of the disclosure includes a method of treating a CCR2-mediated condition or disease comprising administering to a subject an effective amount of any compound or a pharmaceutically acceptable salt thereof as described herein, or a composition as described herein. One aspect of the embodiment includes a method wherein the mediated condition or disease is selected from the group consisting of atherosclerosis, restenosis, multiple sclerosis, inflammatory bowel disease, renal fibrosis, rheumatoid arthritis, obesity, diabetes, chronic obstructive pulmonary disease, idiopathic pulmonary fibrosis, idiopathic pneumonia syndrome, pulmonary fibrosis, transplantation rejection, graft-versus-host disease, cancer, and neuropathic pain.

### DETAILED DESCRIPTION

### General

The present disclosure is directed to compounds and salts thereof, compositions and methods useful in the modulation of chemokine receptor function, particularly CCR2 function. Modulation of chemokine receptor activity, as used herein in its various forms, is intended to encompass antagonism, agonism, partial antagonism, inverse agonism and/or partial agonism of the activity associated with a particular chemokine receptor, preferably the CCR2 receptor. Accordingly, the compounds of the present disclosure are compounds which modulate at least one function or characteristic of mammalian CCR2 function or activity, for example, the function or activity of a a human CCR2 protein. The ability of a compound to modulate the function of CCR2, can be demonstrated in a binding assay (e.g., ligand binding or agonist binding), a migration assay, a signaling assay (e.g., activation of a mammalian G protein, induction of rapid and transient increase in the concentration of cytosolic free calcium), and/or cellular response assay (e.g., stimulation of chemotaxis, exocytosis or inflammatory mediator release by leukocytes).

### Abbreviations and Definitions

When describing the compounds, compositions, methods and processes of this disclosure, the following terms have the following meanings, unless otherwise indicated.

“Alkyl” by itself or as part of another substituent refers to a hydrocarbon group which may be linear, cyclic, or branched or a combination thereof having the number of carbon atoms designated (i.e., C₁₋₈ means one to eight carbon atoms). Examples of alkyl groups include methyl, ethyl, n-propyl, isopropyl, n-butyl, t-butyl, isobutyl, sec-butyl, cyclohexyl, cyclopentyl, (cyclohexyl)methyl, cyclopropylmethyl, bicyclo[2.2.1]heptane, bicyclo[2.2.2]octane, etc. Alkyl groups can be substituted or unsubstituted, unless otherwise indicated. Examples of substituted alkyl include haloalkyl, thioalkyl, aminoalkyl, and the like.

“Alkoxy” refers to —O-alkyl. Examples of an alkoxy group include methoxy, ethoxy, n-propoxy etc.

“Alkenyl” refers to an unsaturated hydrocarbon group which may be linear, cyclic or branched or a combination thereof. Alkenyl groups with 2-8 carbon atoms are preferred. The alkenyl group may contain 1, 2 or 3 carbon-carbon double bonds. Examples of alkenyl groups include ethenyl, n-propenyl, isopropenyl, n-but-2-enyl, n-hex-3-enyl, cyclohexenyl, cyclopentenyl and the like. Alkenyl groups can be substituted or unsubstituted, unless otherwise indicated.

“Alkynyl” refers to an unsaturated hydrocarbon group which may be linear, cyclic or branched or a combination thereof. Alkynyl groups with 2-8 carbon atoms are preferred. The alkynyl group may contain 1, 2 or 3 carbon-carbon triple bonds. Examples of alkynyl groups include ethynyl, n-propynyl, n-but-2-ynyl, n-hex-3-ynyl and the like. Alkynyl groups can be substituted or unsubstituted, unless otherwise indicated.

“Aryl” refers to a polyunsaturated, aromatic hydrocarbon group having a single ring (monocyclic) or multiple rings (bicyclic), which can be fused together or linked covalently. Aryl groups with 6-10 carbon atoms are preferred, where this number of carbon atoms can be designated by C₆₋₁₀, for example. Examples of aryl groups include phenyl and naphthalene-1-yl, naphthalene-2-yl, biphenyl and the like. Aryl groups can be substituted or unsubstituted, unless otherwise indicated.

“Halo” or “halogen”, by itself or as part of a substituent refers to a chlorine, bromine, iodine, or fluorine atom.

“Haloalkyl”, as a substituted alkyl group, refers to a monohaloalkyl or polyhaloalkyl group, most typically substituted with from 1-3 halogen atoms. Examples include 1-chloroethyl, 3-bromopropyl, trifluoromethyl and the like.

“Heterocyclyl” refers to a saturated or unsaturated non-aromatic ring containing at least one heteroatom (typically 1 to 5 heteroatoms) selected from nitrogen, oxygen or sulfur. The heterocyclyl ring may be monocyclic or bicyclic. Preferably, these groups contain 0-5 nitrogen atoms, 0-2 sulfur atoms and 0-2 oxygen atoms. More preferably, these groups contain 0-3 nitrogen atoms, 0-1 sulfur atoms and 0-1 oxygen atoms. Examples of heterocycle groups include pyrrolidine, piperidine, imidazolidine, pyrazolidine, butyrolactam, valerolactam, imidazolidinone, hydantoin, dioxolane, phthalimide, piperidine, 1,4-dioxane, morpholine, thiomorpholine, thiomorpholine-S-oxide, thiomorpholine-S,S-dioxide, piperazine, pyran, pyridone, 3-pyrroline, thiopyran, pyrone, tetrahydrofuran, tetrahydrothiophene, quinuclidine and the like. Preferred heterocyclic groups are monocyclic, though they may be fused or linked covalently to an aryl or heteroaryl ring system.

In one preferred embodiment, heterocyclic groups may be represented by formula (AA) below:

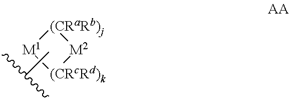

where formula (AA) is attached via a free valence on either M¹ or M²; M¹ represents O, NR^{e}, or S(O)₁; M² represents CR^{f}R^{g}, O, S(O)ₗ, or NR^{e}; l is 0, 1 or 2; j is 1, 2 or 3 and k is 1, 2 or 3, with the proviso that j+k is 3, 4, or 5; and R^{a}, R^{b}, R^{c}, R^{d}, R^{e}, R^{f}, and R^{g} are independently selected from the group consisting of hydrogen, halogen, unsubstituted or substituted C₁₋₈ alkyl, unsubstituted or substituted C₂₋₈ alkenyl, unsubstituted or substituted C₂₋₈ alkynyl, —COR^{h}, —CO₂R^{h}, —CONR^{h}Rⁱ, —NR^{h}CORⁱ, —SO₂R^{h}, —SO₂NR^{h}Rⁱ, —NSO₂R^{h}Rⁱ—NR^{h}Rⁱ, —OR^{h}, —Q¹COR^{h}, —Q¹CO₂R^{h}, —Q¹CONR^{h}Rⁱ, —Q¹NR^{h}CORⁱ, —Q¹SO₂R²⁸, —Q¹SO₂NR^{h}Rⁱ, —Q¹NSO₂R^{h}Rⁱ, —Q¹NR^{h}Rⁱ, —Q¹OR^{h}, wherein Q¹ is a member selected from the group consisting of C₁₋₄ alkylene, C₂₋₄ alkenylene and C₂₋₄ alkynylene, and R^{h} and Rⁱ are independently selected from the group consisting of hydrogen and C₁₋₈ alkyl, and wherein the aliphatic portions of each of the R^{a}, R^{b}, R^{c}, R^{d}, R^{e}, R^{f}, R^{g}, R^{h} and Rⁱ substituents are optionally substituted with from one to three members selected from the group consisting of: halogen, —OH, —ORⁿ, —OC(O)NHRⁿ, —OC(O)NRⁿR^{o}, —SH, —SRⁿ, —S(O)Rⁿ, —S(O)₂Rⁿ, —SO₂NH₂, —S(O)₂NHRⁿ, —S(O)₂NRⁿR^{o}, —NHS(O)₂Rⁿ, —NRⁿS(O)₂R^{o}, —C(O)NH₂, —C(O)NHRⁿ, —C(O)NRⁿR^{o}, —C(O)Rⁿ, —NHC(O)R^{o}, —NRⁿC(O)R^{o}, —NHC(O)NH₂, —NRⁿC(O)NH₂, —NRⁿC(O)NHR^{o}, —NHC(O)NHRⁿ, —NRⁿC(O)NR^{o}, R^{p}, —NHC(O)NRⁿR^{o}, —CO₂H, —CO₂Rⁿ, —NHCO₂Rⁿ, —NRⁿCO₂R^{o}, —CN, —NO₂, —NH₂, —NHRⁿ, —NRⁿR^{o}, —NRⁿS(O)NH₂ and —NRⁿS(O)₂NHR^{o}, wherein Rⁿ, R^{o} and R^{p} are independently an unsubstituted C₁₋₈ alkyl. Additionally, any two of R^{a}, R^{b}, R^{c}, R^{d}, R^{e}, R^{f} and R^{g} may be combined to form a bridged or spirocyclic ring system.

In one preferred embodiment, the number of R^{a}+R^{b}+R^{c}+R^{d} groups that are other than hydrogen is 0, 1 or 2. In a more preferred embodiment, R^{a}, R^{b}, R^{c}, R^{d}, R^{e}, R^{f}, and R^{g} are independently selected from the group consisting of hydrogen, halogen, unsubstituted or substituted C₁₋₈ alkyl, —COR^{h}, —CO₂R^{h}, —CONR^{h}R^{h}, —NR^{h}COR^{h}, —SO₂R^{h}, —SO₂NR^{h}Rⁱ, —NSO₂R^{h}Rⁱ, —NR^{h}Rⁱ, and —ORⁿ, wherein R^{h} and Rⁱ are independently selected from the group consisting of hydrogen and unsubstituted C₁₋₈ alkyl and wherein the aliphatic portions of each of the R^{a}, R^{b}, R^{c}, R^{d}, R^{e}, R^{f} and R^{g} substituents are optionally substituted with from one to three members selected from the group consisting of halogen, —OH, —ORⁿ, —OC(O)NHRⁿ, —OC(O)NRⁿR^{o}, —SH, —SRⁿ, —S(O)R^{o}, —S(O)₂Rⁿ, —SO₂NH₂, —S(O)₂NHRⁿ, —S(O)₂NRⁿR^{o}, —NHS(O)₂Rⁿ, —NRⁿS(O)₂R^{o}, —C(O)NH₂, C(O)NHRⁿ, —C(O)NRⁿR^{o}, —C(O)Rⁿ, —NHC(O)Rⁿ, —NRⁿC(O)R^{o}, —NHC(O)NH₂, —NRⁿC(O)NH₂, —NRⁿC(O)NHR^{o}, —NHC(O)NHRⁿ, —NRⁿC(O)NR^{o}R^{p}, —NHC(O)NRⁿR^{o}, —CO₂H, —CO₂Rⁿ, —NHCO₂Rⁿ, —NRⁿCO₂R^{o}, —CN, —NO₂, —NH₂, —NHRⁿ, —NRⁿR^{o}, —NRⁿS(O)NH₂ and —NRⁿS(O)₂NHR^{o}, wherein Rⁿ, R^{o} and R^{p} are independently an unsubstituted C₁₋₈ alkyl.

In a more preferred embodiment, R^{a}, R^{b}, R^{c}, R^{d}, R^{e}, R^{f}, and R^{g} are independently hydrogen or C₁₋₄ alkyl. In another preferred embodiment, at least three of R^{a}, R^{b}, R^{c}, R^{d}, R^{e}, R^{f}, and R^{g} are hydrogen.

“Heteroaryl” refers to an aromatic group containing at least one heteroatom, where the heteroaryl group may be monocyclic or bicyclic. Examples include pyridyl, pyridazinyl, pyrazinyl, pyrimidinyl, triazinyl, quinolinyl, quinoxalinyl, quinazolinyl, cinnolinyl, phthalazinyl, benzotriazinyl, purinyl, benzimidazolyl, benzopyrazolyl, benzotriazolyl, benzisoxazolyl, isobenzofuryl, isoindolyl, indolizinyl, benzotriazinyl, thienopyridinyl, thienopyrimidinyl, pyrazolopyrimidinyl, imidazopyridines, benzothiazolyl, benzofuranyl, benzothienyl, indolyl, azaindolyl, azaindazolyl, quinolyl, isoquinolyl, isothiazolyl, pyrazolyl, indazolyl, pteridinyl, imidazolyl, triazolyl, tetrazolyl, oxazolyl, isoxazolyl, oxadiazolyl, thiadiazolyl, pyrrolyl, thiazolyl, furyl or thienyl. Preferred heteroaryl groups are those having at least one aryl ring nitrogen atom, such as quinolinyl, quinoxalinyl, purinyl, benzimidazolyl, benzopyrazolyl, benzotriazolyl, benzothiazolyl, indolyl, quinolyl, isoquinolyl and the like. Preferred 6-ring heteroaryl systems include pyridyl, pyridazinyl, pyrazinyl, pyrimidinyl, triazinyl and the like. Preferred 5-ring heteroaryl systems include isothiazolyl, pyrazolyl, imidazolyl, thienyl, furyl, triazolyl, tetrazolyl, oxazolyl, isoxazolyl, oxadiazolyl, thiadiazolyl, pyrrolyl, thiazolyl and the like.

Heterocyclyl and heteroaryl can be attached at any available ring carbon or heteroatom. Each heterocyclyl and heteroaryl may have one or more rings. When multiple rings are present, they can be fused together or linked covalently. Each heterocyclyl and heteroaryl must contain at least one heteroatom (typically 1 to 5 heteroatoms) selected from nitrogen, oxygen or sulfur. Preferably, these groups contain 0-5 nitrogen atoms, 0-2 sulfur atoms and 0-2 oxygen atoms. More preferably, these groups contain 0-3 nitrogen atoms, 0-1 sulfur atoms and 0-1 oxygen atoms. Heterocyclyl and heteroaryl groups can be substituted or unsubstituted, unless otherwise indicated. For substituted groups, the substitution may be on a carbon or heteroatom. For example, when the substitution is oxo (═O or —O⁻), the resulting group may have either a carbonyl (—C(O)—) or a N-oxide (—N⁺—O⁻).

Suitable substituents for substituted alkyl, substituted alkenyl, and substituted alkynyl include halogen, —CN, —CO₂R′, —C(O)R′, —C(O)NR′R″, oxo (═O or —O⁻), —OR′, —OC(O)R′, —OC(O)NR′R″—NO₂, —NR′C(O)R″, —NR′″C(O)NR′R″, —NR′R″, —NR′CO₂R″, —NR′S(O)R″, —NR′S(O)₂R′″, —NR′″S(O)NR′R″, —NR′″S(O)₂NR′R″, —SR′, —S(O)R′, —S(O)₂R′, —S(O)₂NR′R″, —NR′—C(NHR″)═NR′″, —SiR′R″R′″, —N₃, substituted or unsubstituted C₆₋₁₀ aryl, substituted or unsubstituted 5- to 10-membered heteroaryl, and substituted or unsubstituted 3- to 10-membered heterocyclyl. The number of possible substituents range from zero to (2m′+1), where m′ is the total number of carbon atoms in such radical.

Suitable substituents for substituted aryl, substituted heteroaryl and substituted heterocyclyl include halogen, —CN, —CO₂R′, —C(O)R, —C(O)NR′R″, oxo (═O or —O⁻), —OR′, —OC(O)R′, —OC(O)NR′R″, —NO₂, —NR′C(O)R″, —NR′″C(O)NR′R″, —NR′R″, —NR′CO₂R″, —NR′S(O)R″, —NR′S(O)₂R″, —NR′″S(O)NR′R″, —NR′″S(O)₂NR′R″, —SR′, —S(O)R′, —S(O)₂R′, —S(O)₂NR′R″, —NR′—C(NHR″)═NR′″, —SiR′R″R′″, —N₃, substituted or unsubstituted C₁₋₈ alkyl, substituted or unsubstituted C₂₋₈ alkenyl, substituted or unsubstituted C₂₋₈ alkynyl, substituted or unsubstituted C₆₋₁₀ aryl, substituted or unsubstituted 5- to 10-membered heteroaryl, and substituted or unsubstituted 3- to 10-membered heterocyclyl. The number of possible substituents range from zero to the total number of open valences on the aromatic ring system.

As used above, R′, R″ and R′″ each independently refer to a variety of groups including hydrogen, substituted or unsubstituted C₁₋₈ alkyl, substituted or unsubstituted C₂₋₈ alkenyl, substituted or unsubstituted C₂₋₈ alkynyl, substituted or unsubstituted aryl, substituted or unsubstituted heteroaryl, substituted or unsubstituted heterocyclyl, substituted or unsubstituted arylalkyl, substituted or unsubstituted aryloxyalkyl. When R′ and R″ are attached to the same nitrogen atom, they can be combined with the nitrogen atom to form a 3-, 4-, 5-, 6-, or 7-membered ring (for example, —NR′R″ includes 1-pyrrolidinyl and 4-morpholinyl). Furthermore, R′ and R″, R″ and or R′ and R′″ may together with the atom(s) to which they are attached, form a substituted or unsubstituted 5-, 6-, or 7-membered ring.

Two of the substituents on adjacent atoms of an aryl or heteroaryl ring may optionally be replaced with a substituent of the formula -T-C(O)—(CH₂)_{q}-U-, wherein T and U are independently —NR″″—, —O—, —CH₂— or a single bond, and q is an integer of from 0 to 2. Alternatively, two of the substituents on adjacent atoms of the aryl or heteroaryl ring may optionally be replaced with a substituent of the formula -A′-(CH₂)ᵣB′, wherein A′ and B′ are independently —CH₂—, —O—, —NR″″—, —S—, —S(O)—, —S(O)₂—, —S(O)₂NR″″— or a single bond, and r is an integer of from 1 to 3. One of the single bonds of the new ring so formed may optionally be replaced with a double bond. Alternatively, two of the substituents on adjacent atoms of the aryl or heteroaryl ring may optionally be replaced with a substituent of the formula —(CH₂)ₛ—X—(CH₂)ₜ—, where s and t are independently integers of from 0 to 3, and X is —O—, —NR″″—, —S—, —S(O)—, —S(O)₂—, or —S(O)₂NR′—. R″″ in is selected from hydrogen or unsubstituted C₁₋₈ alkyl.

“Heteroatom” is meant to include oxygen (O), nitrogen (N), sulfur (S) and silicon (Si).

“Above natural isotopic abundance” refers to the abundance of isotopes of a chemical element as naturally measured.

“Pharmaceutically acceptable” carrier, diluent, or excipient is a carrier, diluent, or excipient compatible with the other ingredients of the formulation and not deleterious to the recipient thereof.

“Pharmaceutically-acceptable salt” refers to a salt which is acceptable for administration to a patient, such as a mammal (e.g., salts having acceptable mammalian safety for a given dosage regime). Such salts can be derived from pharmaceutically-acceptable inorganic or organic bases and from pharmaceutically-acceptable inorganic or organic acids, depending on the particular substituents found on the compounds described herein. When compounds of the present disclosure contain relatively acidic functionalities, base addition salts can be obtained by contacting the neutral form of such compounds with a sufficient amount of the desired base, either neat or in a suitable inert solvent. Salts derived from pharmaceutically acceptable inorganic bases include aluminum, ammonium, calcium, copper, ferric, ferrous, lithium, magnesium, manganic, manganous, potassium, sodium, zinc and the like. Salts derived from pharmaceutically-acceptable organic bases include salts of primary, secondary, tertiary and quaternary amines, including substituted amines, cyclic amines, naturally-occurring amines and the like, such as arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like. When compounds of the present disclosure contain relatively basic functionalities, acid addition salts can be obtained by contacting the neutral form of such compounds with a sufficient amount of the desired acid, either neat or in a suitable inert solvent. Salts derived from pharmaceutically-acceptable acids include acetic, ascorbic, benzenesulfonic, benzoic, camphosulfonic, citric, ethanesulfonic, fumaric, gluconic, glucoronic, glutamic, hippuric, hydrobromic, hydrochloric, isethionic, lactic, lactobionic, maleic, malic, mandelic, methanesulfonic, mucic, naphthalenesulfonic, nicotinic, nitric, pamoic, pantothenic, phosphoric, succinic, sulfuric, tartaric, p-toluenesulfonic and the like.

“Optionally deuterated alkyl” refers to alkyl groups that may comprise one or more deuterium atoms above natural isotopic abundance.

“Optionally substituted with deuterium” refers to the optional replacement of one or more hydrogen atoms with a corresponding number of deuterium atoms above natural isotopic abundance.

Also included are salts of amino acids such as arginate and the like, and salts of organic acids like glucuronic or galactunoric acids and the like (see, for example, Berge, S. M. et al, “Pharmaceutical Salts”, J. Pharmaceutical Science, 1977, 66:1-19). Certain specific compounds of the present disclosure contain both basic and acidic functionalities that allow the compounds to be converted into either base or acid addition salts.

The neutral forms of the compounds may be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present disclosure.

“Salt thereof” refers to a compound formed when the hydrogen of an acid is replaced by a cation, such as a metal cation or an organic cation and the like. Preferably, the salt is a pharmaceutically-acceptable salt, although this is not required for salts of intermediate compounds which are not intended for administration to a patient.

In addition to salt forms, the present disclosure provides compounds which are in a prodrug form. Prodrugs of the compounds described herein are those compounds that readily undergo chemical changes under physiological conditions to provide the compounds of the present disclosure. Additionally, prodrugs can be converted to the compounds of the present disclosure by chemical or biochemical methods in an ex vivo environment. For example, prodrugs can be slowly converted to the compounds of the present disclosure when placed in a transdermal patch reservoir with a suitable enzyme or chemical reagent.

“Therapeutically effective amount” refers to an amount sufficient to effect treatment when administered to a patient in need of treatment.

“Treating” or “treatment” as used herein refers to the treating or treatment of a disease or medical condition (such as a viral, bacterial or fungal infection or other infectious diseases, as well as autoimmune or inflammatory conditions) in a patient, such as a mammal (particularly a human or a companion animal) which includes ameliorating the disease or medical condition, i.e., eliminating or causing regression of the disease or medical condition in a patient; suppressing the disease or medical condition, i.e., slowing or arresting the development of the disease or medical condition in a patient; or alleviating the symptoms of the disease or medical condition in a patient.

Certain compounds of the present disclosure can exist in unsolvated forms as well as solvated forms, including hydrated forms. In general, both solvated forms and unsolvated forms are intended to be encompassed within the scope of the present disclosure. Certain compounds of the present disclosure may exist in multiple crystalline or amorphous forms (i.e., as polymorphs). In general, all physical forms are equivalent for the uses contemplated by the present disclosure and are intended to be within the scope of the present disclosure.

It will be apparent to one skilled in the art that certain compounds of the present disclosure may exist in tautomeric forms, all such tautomeric forms of the compounds being within the scope of the disclosure. Certain compounds of the present disclosure possess asymmetric carbon atoms (optical centers) or double bonds; the racemates, diastereomers, geometric isomers and individual isomers (e.g., separate enantiomers) are all intended to be encompassed within the scope of the present disclosure. The compounds of the present disclosure may also contain unnatural proportions of atomic isotopes at one or more of the atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the present disclosure, whether radioactive or not, are intended to be encompassed within the scope of the present disclosure.

Compounds that Modulate CCR2 Activity

The present disclosure provides deuterated compounds that modulate CCR2 activity. Chemokine receptors are integral membrane proteins which interact with an extracellular ligand, such as a chemokine, and mediate a cellular response to the ligand, e.g., chemotaxis, increased intracellular calcium ion concentration, etc. Therefore, modulation of a chemokine receptor function, e.g., interference with a chemokine receptor ligand interaction, will modulate a chemokine receptor mediated response, and treat or prevent a chemokine receptor mediated condition or disease. Modulation of a chemokine receptor function includes both inducement and inhibition of the function. The type of modulation accomplished will depend on the characteristics of the compound, i.e., antagonist or full, partial or inverse agonist.

Without intending to be bound by any particular theory, it is believed that the compounds provided herein interfere with the interaction between a chemokine receptor and one or more cognate ligands. In particular, it is believed that the compounds interfere with the interaction between CCR2 and a CCR2 ligand, such as MCP-1. Compounds contemplated by the disclosure include, but are not limited to, the exemplary compounds provided herein and salts thereof.

For example, compounds of this disclosure act as potent CCR2 antagonists, and this antagonistic activity has been further confirmed in animal testing for inflammation, one of the hallmark disease states for CCR2. Accordingly, the compounds provided herein are useful in pharmaceutical compositions, methods for the treatment of CCR2-mediated diseases, and as controls in assays for the identification of competitive CCR2 antagonists.

In one embodiment, the compounds of the present disclosure are represented by formula (I), or salts thereof:

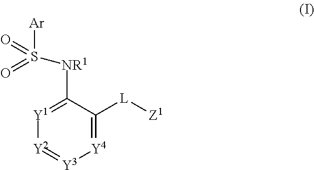

Ar is selected from the group consisting of substituted or unsubstituted optionally deuterated C₆₋₁₀ aryl and substituted or unsubstituted optionally deuterated 5- to 10-membered heteroaryl.

R¹ is selected from the group consisting of hydrogen or detuerium, substituted or unsubstituted C₁₋₈ alkyl, substituted or unsubstituted optionally deuterated C₂₋₆ alkenyl, substituted or unsubstituted optionally deuterated C₂₋₆ alkynyl, and substituted or unsubstituted optionally deuterated 3- to 10-membered heterocyclyl;

Y¹ is selected from the group consisting of —CR^{2a}—, —N—, and —N⁺(O)⁻—;

Y² is selected from the group consisting of —CR^{2b}—, —N—, and —N⁺(O)⁻—;

Y³ is selected from the group consisting of —CR^{2c}—, —N—, and —N⁺(O)⁻—;

R^{2a}, R^{2b} and R^{2c} are each independently selected from the group consisting of hydrogen or deuterium, halogen, —CN, —C(O)R³, —CO₂R³, —C(O)NR³R⁴, —OR³, —OC(O)R³, —OC(O)NR³R⁴, —SR³, —S(O)R³, —S(O)₂R³, —S(O)₂NR³R⁴, —NO₂, —NR³R⁴, —NR³C(O)R⁴, —NR³C(O)OR⁴, —NR³S(O)₂R⁴, —NR³C(O)NR⁴R⁵, substituted or unsubstituted optionally deuterated C₁₋₈ alkyl, substituted or unsubstituted optionally deuterated C₂₋₈ alkenyl, substituted or unsubstituted optionally deuterated C₂₋₈ alkynyl, substituted or unsubstituted optionally deuterated 3- to 10-membered heterocyclyl, substituted or unsubstituted optionally deuterated C₆₋₁₀ aryl, and substituted or unsubstituted optionally deuterated 5- to 10-membered heteroaryl;

R³, R⁴, and R⁵ are each independently selected from the group consisting of hydrogen or deuterium, substituted or unsubstituted optionally deuterated C₁₋₈ alkyl, substituted or unsubstituted optionally deuterated C₂₋₈ alkenyl, substituted or unsubstituted optionally deuterated C₂₋₈ alkynyl, substituted or unsubstituted optionally deuterated C₆₋₁₀ aryl, substituted or unsubstituted optionally deuterated 5- to 10-membered heteroaryl, and substituted or unsubstituted optionally deuterated 3- to 10-membered heterocyclyl;

R³ and R⁴, R⁴ and R⁵ or R³ and R⁵ may, together with the atoms to which they are attached, form a substituted or unsubstituted optionally deuterated 5-, 6-, or 7-membered ring;

L is selected from the group consisting of a bond, —O—, —S—, —S(O)—, —S(O)₂—, —CR⁶R⁷—, —C(═N—O—R⁹)—, —C(O)NR⁸—, and —NR⁸C(O)—;

R⁶ and R⁷ are each independently selected from the group consisting of hydrogen or deuterium, halogen, substituted or unsubstituted optionally deuterated C₁₋₈ alkyl, substituted or unsubstituted optionally deuterated 3- to 10-membered heterocyclyl, substituted or unsubstituted optionally deuterated C₂₋₆ alkenyl, substituted or unsubstituted optionally deuterated C₂₋₆ alkynyl, —CN, —OR⁹, —NR¹⁰R¹¹, —S(O)R⁹, and —S(O)₂R⁹;

R⁶ and R⁷ may, together with the carbon atom to which they are attached, form substituted or unsubstituted optionally deuterated C₃₋₈ cycloalkyl or substituted or unsubstituted optionally deuterated 3- to 10-membered heterocyclic ring;

R⁹ is selected from the group consisting of hydrogen or deuterium, substituted or unsubstituted optionally deuterated C₁₋₈ alkyl, substituted or unsubstituted optionally deuterated C₂₋₈ alkenyl, substituted or unsubstituted optionally deuterated C₂₋₈ alkynyl, substituted or unsubstituted optionally deuterated C₆₋₁₀ aryl, substituted or unsubstituted optionally deuterated 5- to 10-membered heteroaryl, and substituted or unsubstituted optionally deuterated 3- to 10-membered heterocyclyl;

R¹⁰ and R¹¹ are each independently selected from the group consisting of substituted or unsubstituted optionally deuterated C₁₋₈ alkyl, substituted or unsubstituted optionally deuterated 3- to 10-membered heterocyclyl, substituted or unsubstituted optionally deuterated C₆₋₁₀ aryl, substituted or unsubstituted optionally deuterated 5- to 10-membered heteroaryl, substituted or unsubstituted optionally deuterated C₂₋₈ alkenyl, and substituted or unsubstituted optionally deuterated C₂₋₈ alkynyl;

R¹⁰ and R¹¹ of —NR¹⁰R¹¹ may, together with the nitrogen, form substituted or unsubstituted optionally deuterated 3- to 10-membered heterocyclyl;

R⁸ is selected from the group consisting of hydrogen or deuterium, —C(O)R¹², —S(O)₂R¹², —CO₂R¹², substituted or unsubstituted optionally deuterated C₁₋₈ alkyl, substituted or unsubstituted optionally deuterated 3- to 10-membered heterocyclyl, substituted or unsubstituted optionally deuterated C₂₋₆ alkenyl, and substituted or unsubstituted optionally deuterated C₂₋₆ alkynyl;

R¹² is selected from the group consisting of substituted or unsubstituted optionally deuterated C₁₋₈ alkyl, substituted or unsubstituted optionally deuterated C₂₋₆ alkenyl, substituted or unsubstituted optionally deuterated C₂₋₆ alkynyl, substituted or unsubstituted optionally deuterated 3- to 10-membered heterocyclyl, substituted or unsubstituted optionally deuterated C₆₋₁₀ aryl, and substituted or unsubstituted optionally deuterated 5- to 10-membered heteroaryl;

Z¹ is selected from the group consisting of substituted or unsubstituted optionally deuterated C6-10 aryl, substituted or unsubstituted optionally deuterated 5- to 10-membered heteroaryl, substituted or unsubstituted optionally deuterated 3- to 10-membered heterocyclyl, and —NR¹³R¹⁴;

R¹³ and R¹⁴ are each independently selected from the group consisting of hydrogen or deuterium, substituted or unsubstituted optionally deuterated C₁₋₈ alkyl, substituted or unsubstituted C₂₋₈ alkenyl, substituted or unsubstituted optionally deuterated C₂₋₈ alkynyl, substituted or unsubstituted optionally deuterated 3- to 10-membered heterocyclyl, substituted or unsubstituted optionally deuterated C₆₋₁₀ aryl, substituted or unsubstituted optionally deuterated 5- to 10-membered heteroaryl, substituted or unsubstituted optionally deuterated (C₁₋₄ alkyl)-(C₆₋₁₀ aryl), and substituted or unsubstituted optionally deuterated (C₁₋₄ alkyl)-(5- to 10-membered heteroaryl);

R¹³ and R¹⁴ may, together with the nitrogen, form a substituted or unsubstituted optionally deuterated 4-, 5-, 6-, or 7-membered heterocyclyl.

Y⁴ is selected from the group consisting of —N— and —N⁺(O)⁻—;

wherein the compound comprises at least one deuterium above natural isotopic abundance, or a pharmaceutically acceptable salt thereof.

In one embodiment, at least one of Y¹, Y², Y³ or Z¹ comprises deuterium above natural isotopic abundance.

In one embodiment, the compounds of the present disclosure are represented by formula (II), or salts thereof:

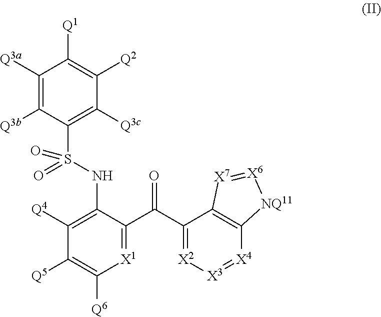

each of Q¹ and Q² independently is hydrogen, deuterium, halogen, optionally deuterated C₁₋₈ alkyl, —CN, or optionally deuterated C₁₋₈ haloalkyl;

Q⁴ is hydrogen or deuterium;

Q⁵ is halogen or optionally deuterated C₁₋₈ alkyl;

Q⁶ is hydrogen or deuterium;

each of X¹, X², X⁴, X⁶, and X⁷ independently is —CQ⁷—, —N—, or —NO—;

X³ is —CQ⁷—;

each Q⁷ independently is hydrogen or deuterium, halogen, substituted or unsubstituted optionally deuterated C₁₋₈ alkyl, substituted or unsubstituted optionally deuterated C₂₋₈ alkenyl, substituted or unsubstituted optionally deuterated C₂₋₈ alkynyl, —CN, ═O, —NO₂, —OQ⁸, —OC(O)Q⁸, —CO₂Q⁸,—C(O)Q⁸, —C(O)NQ⁹Q⁸, —OC(O)NQ⁹Q⁸, —NQ¹⁰C(O)Q⁸, —NQ¹⁰C(O)NQ⁹Q⁸, —NQ⁹Q⁸, —NQ¹⁰CO₂Q⁸, —SQ⁸, —S(O)Q⁸, —S(O)₂Q⁸, —S(O)₂NQ⁹Q⁸, —NQ¹⁰S(O)₂Q⁸, substituted or unsubstituted optionally deuterated C₆₋₁₀ aryl, substituted or unsubstituted optionally deuterated 5- to 10-membered heteroaryl and substituted or unsubstituted optionally deuterated 3- to 10-membered heterocyclyl;

each occurrence of Q⁸, Q⁹, and Q¹⁰ is independently selected from the group consisting of hydrogen or deuterium, optionally deuterated C₁₋₈ alkyl, optionally deuterated C₂₋₈ alkenyl, optionally deuterated C₂₋₈ alkynyl, optionally deuterated aryl, or optionally deuterated heteroaryl; or Q⁹ and Q⁸ or Q¹⁰ and Q⁸, together with the atom(s) to which they are attached, form an substituted or unsubstituted optionally deuterated 5-, 6-, or 7-membered ring;

Q¹¹ is selected from the group consisting of hydrogen, optionally deuterated C₁₋₈ alkyl, optionally deuterated C₂₋₈ alkenyl, optionally deuterated C₂₋₈ alkynyl, optionally deuterated substituted or unsubstituted C₆₋₁₀ aryl, optionally deuterated substituted or unsubstituted 5-to 10-membered heteroaryl, and optionally deuterated substituted or unsubstituted 3- to 10-membered heterocycle; and wherein the compound comprises at least one deuterium above natural isotopic abundance, or a pharmaceutically acceptable salt thereof.

In as much as any composition described herein as Formulas I and II and defines an atom as nitrogen in the context of an aromatic ring, the person of ordinary skill in the art should understand that the nitrogen atom would maintain its aromaticity with adjacent carbon and nitrogen atoms. Nothing in this disclosure should be construed otherwise.

In one embodiment the compound of Formula II, at least one of Q¹ or Q² is other than hydrogen. In one embodiment, each Q^{a3}, Q^{3b} and Q^{3c} independently is hydrogen or deuterium. In one embodiment, one or more Q⁴, Q⁵, Q⁶, or Q⁷ comprises deuterium above natural isotopic abundance. In one embodiment, wherein Q¹ is halogen. In one embodiment, Q² is deuterated C₁₋₈ haloalkyl. In one embodiment, Q² is deuterated C₁₋₈ haloalkyl. In one embodiment, Q² is C₁₋₈ haloalkyl. In one embodiment, Q⁴ is deuterium. In one embodiment, Q⁴ is hydrogen. In one embodiment, Q⁵ is deuterated C₁₋₈ alky. In one embodiment, Q⁵ is C₁₋₈ alkyl. In one embodiment, Q⁶ is deuterium. In one embodiment, Q⁶ is hydrogen. In one embodiment, Q⁷ is deuterium. In one embodiment, Q⁷ is hydrogen.

In one embodiment, the compounds of the present disclosure are represented by formula (II), or salts thereof:

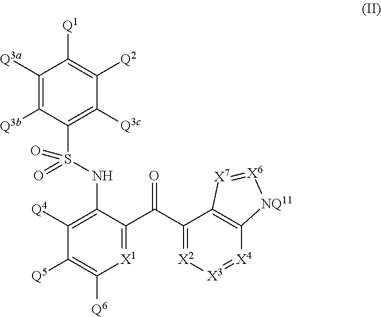

wherein each of Q¹ and Q² independently is hydrogen, deuterium, halogen, C₁₋₈ alkyl, —CN, or C₁₋₈ haloalkyl;

each Q³ independently is deuterium or hydrogen;

Q⁴ is hydrogen or deuterium;

Q⁵ is halogen or optionally deuterated C₁₋₈ alkyl;

Q⁶ is hydrogen or deuterium;

each of X¹, X², X⁴, X⁶, and X⁷ independently is —CQ⁷—, —N—, or —NO—;

X³ is —CQ⁷—;

each Q⁷ independently is hydrogen or deuterium;

Q¹¹ is hydrogen; and

wherein the compound comprises at least one deuterium above natural isotopic abundance, or a pharmaceutically acceptable salt thereof.

In one embodiment, at least one of Q¹ or Q² is other than hydrogen. In one embodiment, one or more of Q⁴, Q⁵, Q⁶, or Q⁷ comprises deuterium above natural isotopic abundance.

In one embodiment the compounds of the present disclosure are compounds selected from the group consisting of:

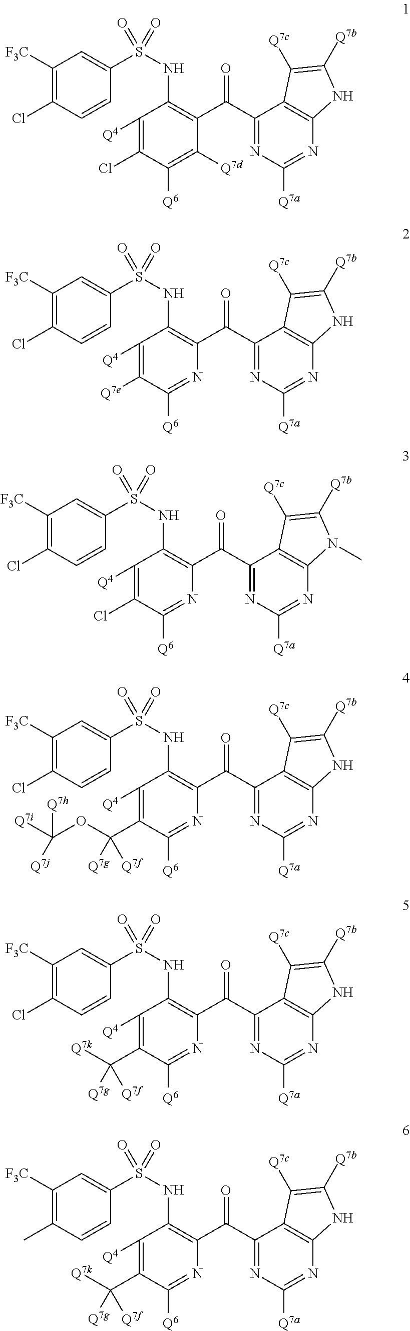

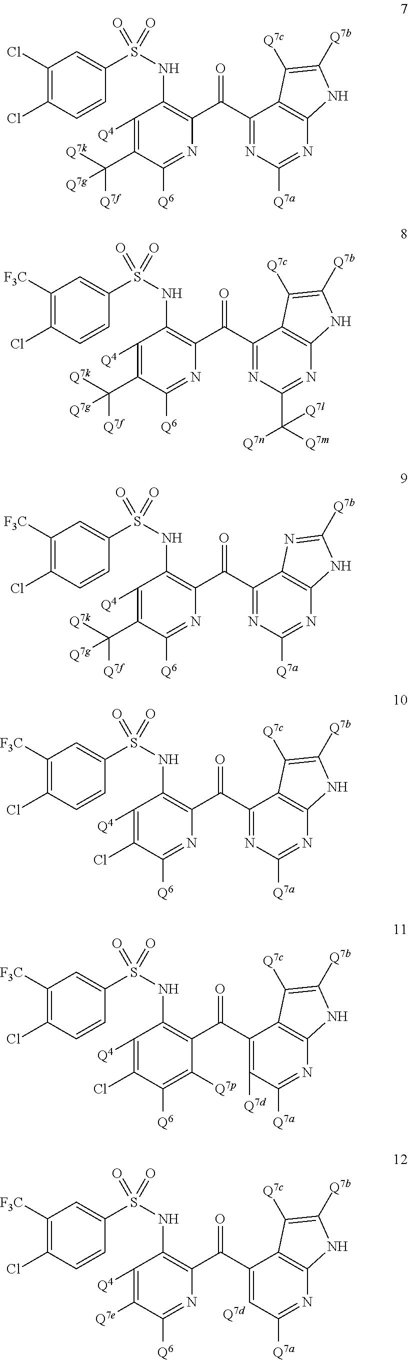

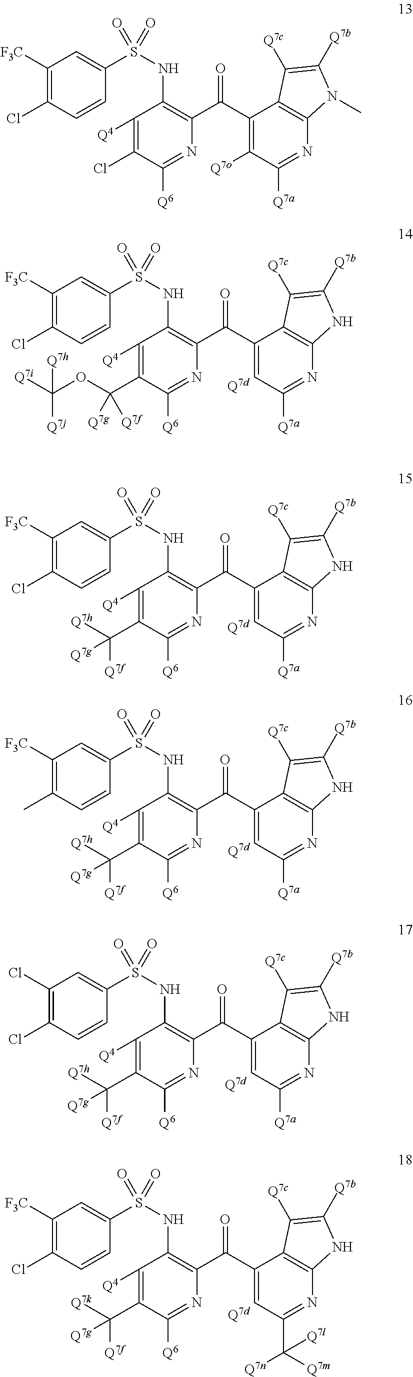

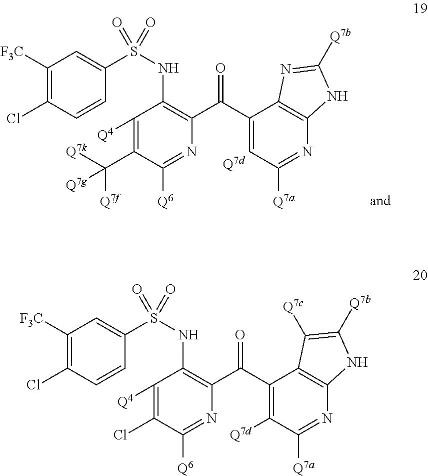

or a pharmaceutically acceptable salt thereof wherein each Q⁴ is independently hydrogen or deuterium;

each Q⁶ is independently hydrogen or deuterium;

each Q^{7a}, Q^{7b}, Q^{7c}, Q^{7d}, Q^{7e}, Q^{7f}, Q^{7g}, Q^{7h}, Q⁷ⁱ, Q^{7j}, Q^{7k}, Q^{7l}, Q^{7m}, Q⁷ⁿ, Q^{7o} independently is hydrogen or deuterium; and

wherein the compound comprises at least one deuterium above natural isotopic abundance.

In one embodiment, one or more of Q⁴, Q⁶, or Q^{7a-o} comprises deuterium above natural isotopic abundance. In one embodiment for each compound, or a pharmaceutically acceptable salt thereof, the compound comprises one deuterium above natural isotopic abundance. In one embodiment, the compound comprises two deuterium atoms above natural isotopic abundance. In one embodiment, the compound comprises three deuterium atoms above natural isotopic abundance. In one embodiment, each position represented as deuterium has deuterium enrichment of at least 50%. In one embodiment, each position represented as deuterium has deuterium enrichment of at least 90%.

In one embodiment, the compounds of the present disclosure are compounds selected from the group consisting of:

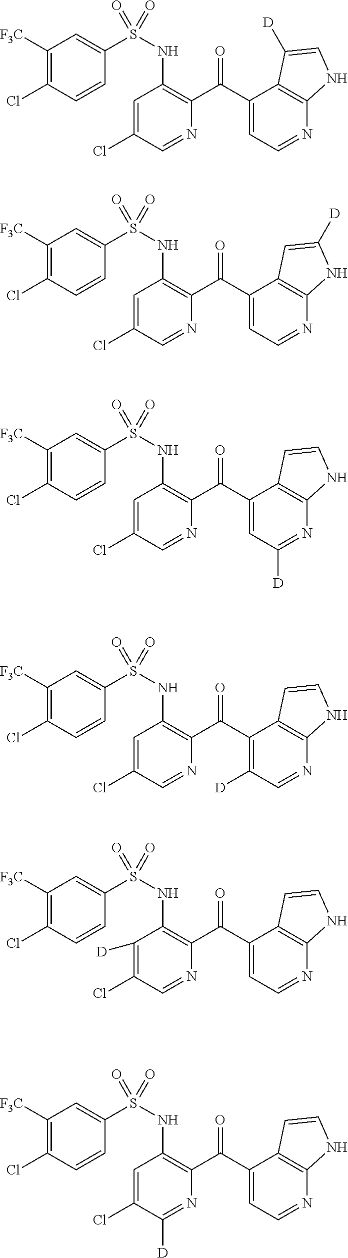 [SMILES: [2H]c1c[nH]c2nccc(C(=O)c3ncc(Cl)cc3NS(=O)(=O)c3ccc(Cl)c(C(F)(F)F)c3)c12 ; [2H]c1cc2c(C(=O)c3ncc(Cl)cc3NS(=O)(=O)c3ccc(Cl)c(C(F)(F)F)c3)ccnc2[nH]1 ; [2H]c1cc(C(=O)c2ncc(Cl)cc2NS(=O)(=O)c2ccc(Cl)c(C(F)(F)F)c2)c2cc[nH]c2n1 ; [2H]c1cnc2[nH]ccc2c1C(=O)c1ncc(Cl)cc1NS(=O)(=O)c1ccc(Cl)c(C(F)(F)F)c1 ; [2H]c1c(Cl)cnc(C(=O)c2ccnc3[nH]ccc23)c1NS(=O)(=O)c1ccc(Cl)c(C(F)(F)F)c1 ; [2H]c1nc(C(=O)c2ccnc3[nH]ccc23)c(NS(=O)(=O)c2ccc(Cl)c(C(F)(F)F)c2)cc1Cl]

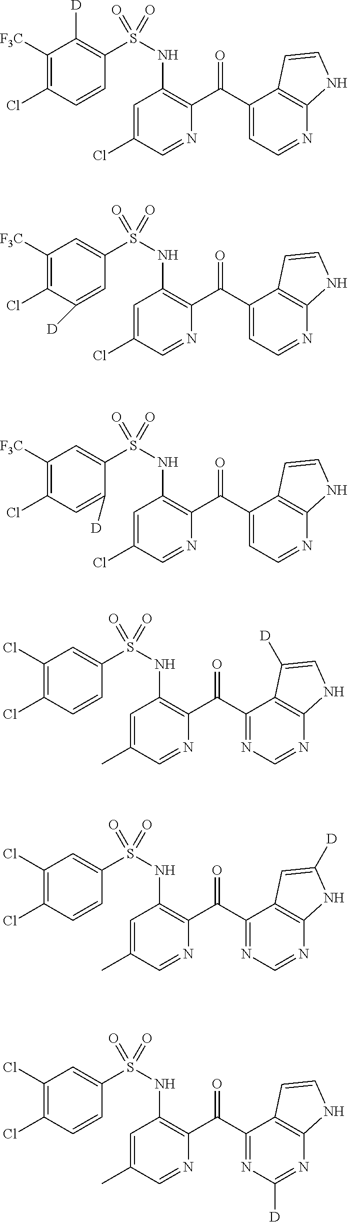 [SMILES: [2H]c1c(S(=O)(=O)Nc2cc(Cl)cnc2C(=O)c2ccnc3[nH]ccc23)ccc(Cl)c1C(F)(F)F ; [2H]c1cc(S(=O)(=O)Nc2cc(Cl)cnc2C(=O)c2ccnc3[nH]ccc23)cc(C(F)(F)F)c1Cl ; [2H]c1cc(Cl)c(C(F)(F)F)cc1S(=O)(=O)Nc1cc(Cl)cnc1C(=O)c1ccnc2[nH]ccc12 ; [2H]c1c[nH]c2ncnc(C(=O)c3ncc(C)cc3NS(=O)(=O)c3ccc(Cl)c(Cl)c3)c12 ; [2H]c1cc2c(C(=O)c3ncc(C)cc3NS(=O)(=O)c3ccc(Cl)c(Cl)c3)ncnc2[nH]1 ; [2H]c1nc(C(=O)c2ncc(C)cc2NS(=O)(=O)c2ccc(Cl)c(Cl)c2)c2cc[nH]c2n1]

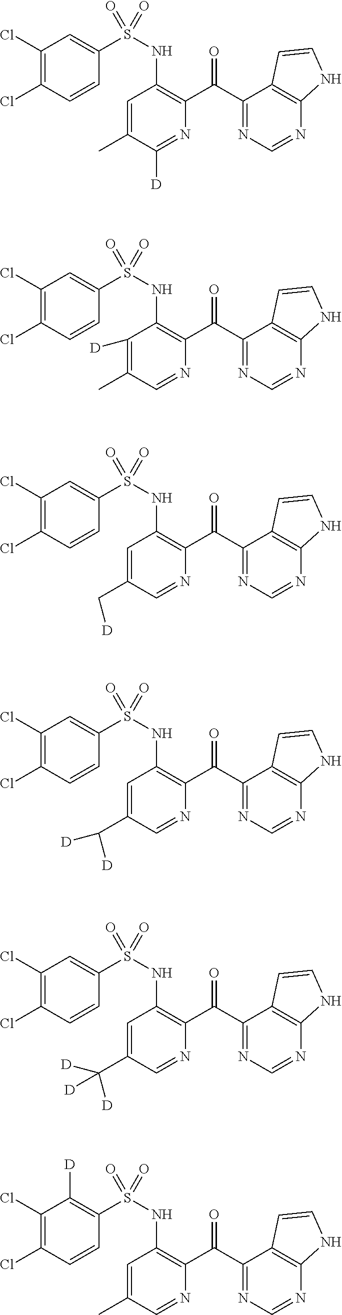 [SMILES: [2H]c1nc(C(=O)c2ncnc3[nH]ccc23)c(NS(=O)(=O)c2ccc(Cl)c(Cl)c2)cc1C ; [2H]c1c(C)cnc(C(=O)c2ncnc3[nH]ccc23)c1NS(=O)(=O)c1ccc(Cl)c(Cl)c1 ; [2H]Cc1cnc(C(=O)c2ncnc3[nH]ccc23)c(NS(=O)(=O)c2ccc(Cl)c(Cl)c2)c1 ; [2H]C([2H])c1cnc(C(=O)c2ncnc3[nH]ccc23)c(NS(=O)(=O)c2ccc(Cl)c(Cl)c2)c1 ; [2H]C([2H])([2H])c1cnc(C(=O)c2ncnc3[nH]ccc23)c(NS(=O)(=O)c2ccc(Cl)c(Cl)c2)c1 ; [2H]c1c(S(=O)(=O)Nc2cc(C)cnc2C(=O)c2ncnc3[nH]ccc23)ccc(Cl)c1Cl]

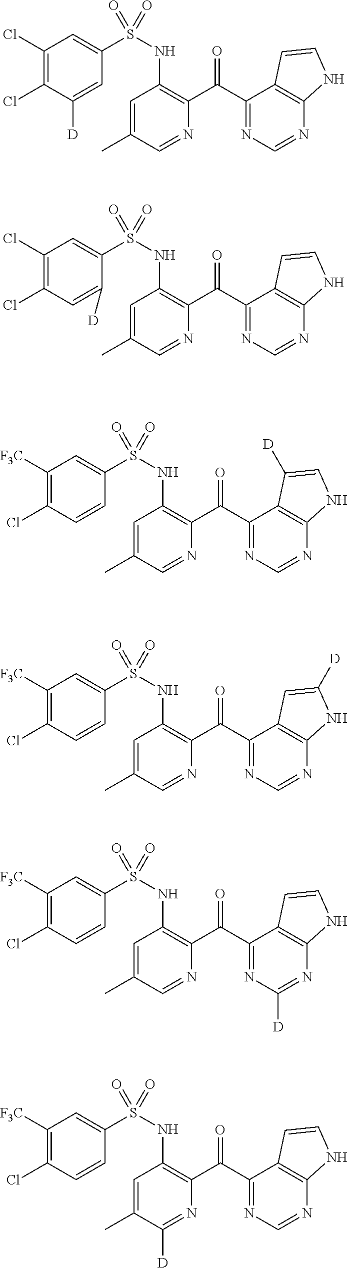 [SMILES: [2H]c1cc(S(=O)(=O)Nc2cc(C)cnc2C(=O)c2ncnc3[nH]ccc23)cc(Cl)c1Cl ; [2H]c1cc(Cl)c(Cl)cc1S(=O)(=O)Nc1cc(C)cnc1C(=O)c1ncnc2[nH]ccc12 ; [2H]c1c[nH]c2ncnc(C(=O)c3ncc(C)cc3NS(=O)(=O)c3ccc(Cl)c(C(F)(F)F)c3)c12 ; [2H]c1cc2c(C(=O)c3ncc(C)cc3NS(=O)(=O)c3ccc(Cl)c(C(F)(F)F)c3)ncnc2[nH]1 ; [2H]c1nc(C(=O)c2ncc(C)cc2NS(=O)(=O)c2ccc(Cl)c(C(F)(F)F)c2)c2cc[nH]c2n1 ; [2H]c1nc(C(=O)c2ncnc3[nH]ccc23)c(NS(=O)(=O)c2ccc(Cl)c(C(F)(F)F)c2)cc1C]

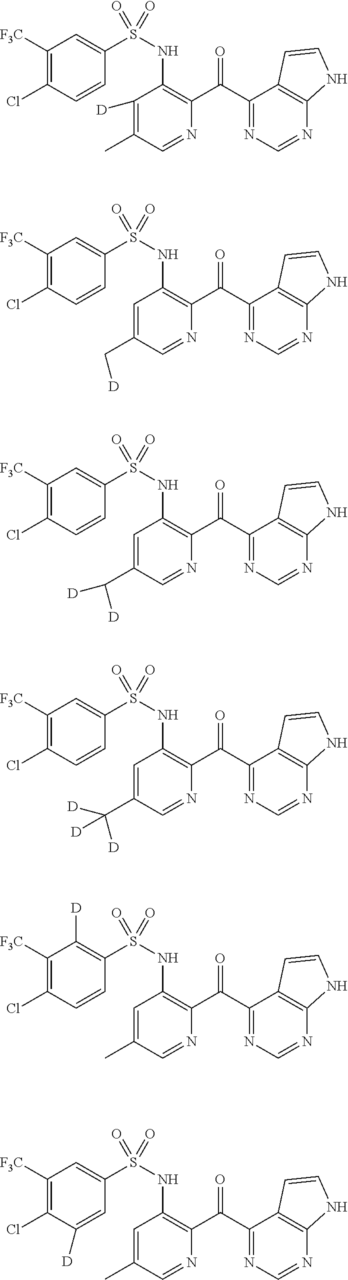 [SMILES: [2H]c1c(C)cnc(C(=O)c2ncnc3[nH]ccc23)c1NS(=O)(=O)c1ccc(Cl)c(C(F)(F)F)c1 ; [2H]Cc1cnc(C(=O)c2ncnc3[nH]ccc23)c(NS(=O)(=O)c2ccc(Cl)c(C(F)(F)F)c2)c1 ; [2H]C([2H])c1cnc(C(=O)c2ncnc3[nH]ccc23)c(NS(=O)(=O)c2ccc(Cl)c(C(F)(F)F)c2)c1 ; [2H]C([2H])([2H])c1cnc(C(=O)c2ncnc3[nH]ccc23)c(NS(=O)(=O)c2ccc(Cl)c(C(F)(F)F)c2)c1 ; [2H]c1c(S(=O)(=O)Nc2cc(C)cnc2C(=O)c2ncnc3[nH]ccc23)ccc(Cl)c1C(F)(F)F ; [2H]c1cc(S(=O)(=O)Nc2cc(C)cnc2C(=O)c2ncnc3[nH]ccc23)cc(C(F)(F)F)c1Cl]

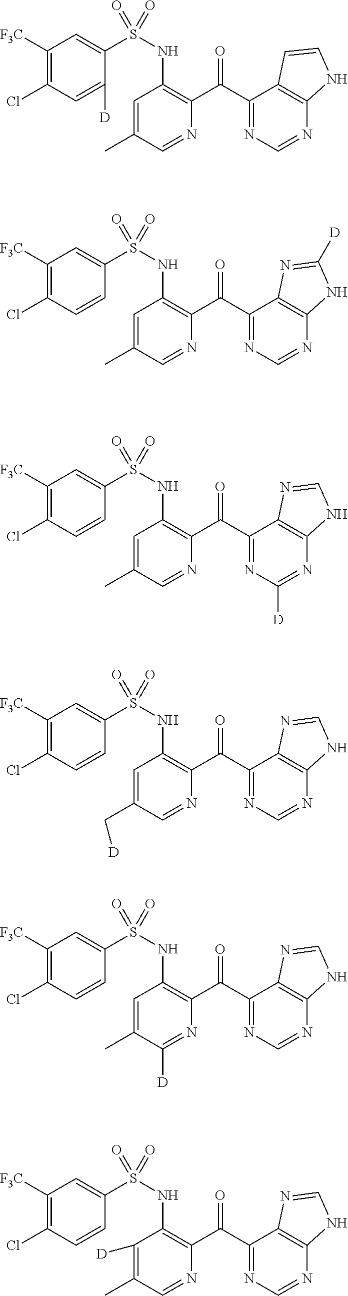 [SMILES: [2H]c1cc(Cl)c(C(F)(F)F)cc1S(=O)(=O)Nc1cc(C)cnc1C(=O)c1ncnc2[nH]ccc12 ; [2H]c1nc2c(C(=O)c3ncc(C)cc3NS(=O)(=O)c3ccc(Cl)c(C(F)(F)F)c3)ncnc2[nH]1 ; [2H]c1nc(C(=O)c2ncc(C)cc2NS(=O)(=O)c2ccc(Cl)c(C(F)(F)F)c2)c2nc[nH]c2n1 ; [2H]Cc1cnc(C(=O)c2ncnc3[nH]cnc23)c(NS(=O)(=O)c2ccc(Cl)c(C(F)(F)F)c2)c1 ; [2H]c1nc(C(=O)c2ncnc3[nH]cnc23)c(NS(=O)(=O)c2ccc(Cl)c(C(F)(F)F)c2)cc1C ; [2H]c1c(C)cnc(C(=O)c2ncnc3[nH]cnc23)c1NS(=O)(=O)c1ccc(Cl)c(C(F)(F)F)c1]

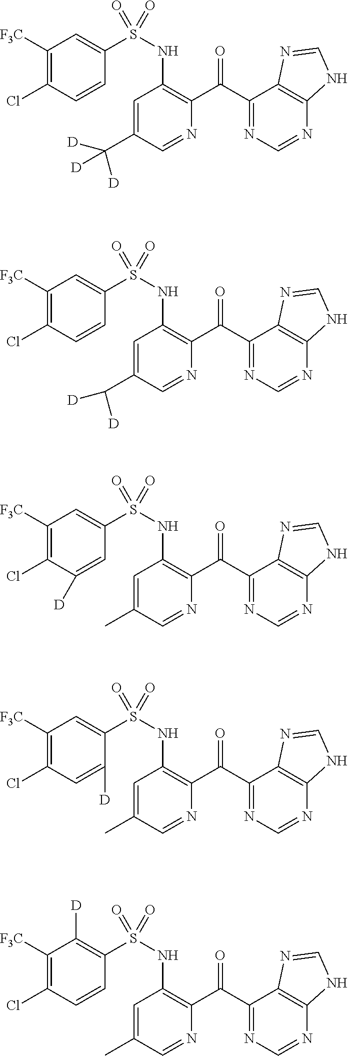 [SMILES: [2H]C([2H])([2H])c1cnc(C(=O)c2ncnc3[nH]cnc23)c(NS(=O)(=O)c2ccc(Cl)c(C(F)(F)F)c2)c1 ; [2H]C([2H])c1cnc(C(=O)c2ncnc3[nH]cnc23)c(NS(=O)(=O)c2ccc(Cl)c(C(F)(F)F)c2)c1 ; [2H]c1cc(S(=O)(=O)Nc2cc(C)cnc2C(=O)c2ncnc3[nH]cnc23)cc(C(F)(F)F)c1Cl ; [2H]c1cc(Cl)c(C(F)(F)F)cc1S(=O)(=O)Nc1cc(C)cnc1C(=O)c1ncnc2[nH]cnc12 ; [2H]c1c(S(=O)(=O)Nc2cc(C)cnc2C(=O)c2ncnc3[nH]cnc23)ccc(Cl)c1C(F)(F)F]

or a pharmaceutically acceptable salt thereof.

In one embodiment, a pharmaceutical composition comprising any of the compound described herein or a pharmaceutically acceptable salt thereof and a pharmaceutically acceptable carrier.

One embodiment of the disclosure includes a method of treating a CCR2-mediated condition or disease comprising administering to a subject an effective amount of any compound or a pharmaceutically acceptable salt thereof as described herein, or a composition as described herein. One aspect of the embodiment includes a method wherein the mediated condition or disease is selected from the group consisting of atherosclerosis, restenosis, multiple sclerosis, inflammatory bowel disease, renal fibrosis, rheumatoid arthritis, obesity, diabetes, chronic obstructive pulmonary disease, idiopathic pulmonary fibrosis, idiopathic pneumonia syndrome, pulmonary fibrosis, transplantation rejection, graft-versus-host disease, cancer, and neuropathic pain.

Compositions that Modulate Chemokine Activity

In another aspect, the present disclosure provides compositions that modulate chemokine activity, specifically CCR2 activity. Generally, the compositions for modulating chemokine receptor activity in humans and animals will comprise a pharmaceutically acceptable excipient or diluent and a compound having the formula provided above as formula (I).

The term “composition” as used herein is intended to encompass a product comprising the specified ingredients in the specified amounts, as well as any product which results, directly or indirectly, from combination of the specified ingredients in the specified amounts. By “pharmaceutically acceptable” it is meant the carrier, diluent or excipient must be compatible with the other ingredients of the formulation and not deleterious to the recipient thereof.

The pharmaceutical compositions for the administration of the compounds of this disclosure may conveniently be presented in unit dosage form and may be prepared by any of the methods well known in the art of pharmacy. All methods include the step of bringing the active ingredient into association with the carrier which constitutes one or more accessory ingredients. In general, the pharmaceutical compositions are prepared by uniformly and intimately bringing the active ingredient into association with a liquid carrier or a finely divided solid carrier or both, and then, if necessary, shaping the product into the desired formulation. In the pharmaceutical composition the active object compound is included in an amount sufficient to produce the desired effect upon the process or condition of diseases.

The pharmaceutical compositions containing the active ingredient may be in a form suitable for oral use, for example, as tablets, troches, lozenges, aqueous or oily suspensions, dispersible powders or granules, emulsions and self emulsifications as described in U.S. Pat. No. 6,451,339, hard or soft capsules, or syrups or elixirs. Compositions intended for oral use may be prepared according to any method known to the art for the manufacture of pharmaceutical compositions. Such compositions may contain one or more agents selected from sweetening agents, flavoring agents, coloring agents and preserving agents in order to provide pharmaceutically elegant and palatable preparations. Tablets contain the active ingredient in admixture with other non-toxic pharmaceutically acceptable excipients which are suitable for the manufacture of tablets. These excipients may be, for example, inert diluents such as cellulose, silicon dioxide, aluminum oxide, calcium carbonate, sodium carbonate, glucose, mannitol, sorbitol, lactose, calcium phosphate or sodium phosphate; granulating and disintegrating agents, for example, corn starch, or alginic acid; binding agents, for example PVP, cellulose, PEG, starch, gelatin or acacia, and lubricating agents, for example magnesium stearate, stearic acid or talc. The tablets may be uncoated or they may be coated enterically or otherwise by known techniques to delay disintegration and absorption in the gastrointestinal tract and thereby provide a sustained action over a longer period. For example, a time delay material such as glyceryl monostearate or glyceryl distearate may be employed. They may also be coated by the techniques described in the U.S. Pat. Nos. 4,256,108; 4,166,452; and 4,265,874 to form osmotic therapeutic tablets for control release.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin, or olive oil. Additionally, emulsions can be prepared with a non-water miscible ingredient such as oils and stabilized with surfactants such as mono-diglycerides, PEG esters and the like.

Aqueous suspensions contain the active materials in admixture with excipients suitable for the manufacture of aqueous suspensions. Such excipients are suspending agents, for example sodium carboxymethylcellulose, methylcellulose, hydroxypropylmethylcellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate, one or more coloring agents, one or more flavoring agents, and one or more sweetening agents, such as sucrose or saccharin.

Oily suspensions may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

The pharmaceutical compositions of the disclosure may also be in the form of oil in water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol or sucrose. Such formulations may also contain one or more of a demulcent, a preservative, a flavoring agent, and coloring agent. Oral solutions can be prepared in combination with, for example, cyclodextrin, PEG and surfactants.

The pharmaceutical compositions may be in the form of a sterile injectable aqueous or oleaginous suspension. This suspension may be formulated according to the known art using those suitable dispersing or wetting agents and suspending agents which have been mentioned above. The sterile injectable preparation may also be a sterile injectable solution or suspension in a non toxic parenterally acceptable diluent or solvent, for example as a solution in 1,3-butane diol. Among the acceptable vehicles and solvents that may be employed are water, Ringer's solution and isotonic sodium chloride solution. In addition, sterile, axed oils are conventionally employed as a solvent or suspending medium. For this purpose any bland fixed oil may be employed including synthetic mono- or diglycerides. In addition, fatty acids such as oleic acid find use in the preparation of injectables.

The compounds of the present disclosure may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials are cocoa butter and polyethylene glycols. Additionally, the compounds can be administered via ocular delivery by means of solutions or ointments. Still further, transdermal delivery of the subject compounds can be accomplished by means of iontophoretic patches and the like.

For topical use, creams, ointments, jellies, solutions or suspensions containing the compounds of the present disclosure are employed. As used herein, topical application is also meant to include the use of mouth washes and gargles.

The pharmaceutical compositions and methods of the present disclosure may further comprise other therapeutically active compounds as noted herein, such as those applied in the treatment of the above mentioned pathological conditions.

In one embodiment, the present disclosure provides a composition consisting of a pharmaceutically acceptable carrier and a compound of the disclosure.

### Methods of Treatment

Depending on the disease to be treated and the subject's condition, the compounds and compositions of the present disclosure may be administered by oral, parenteral (e.g., intramuscular, intraperitoneal, intravenous, ICV, intracisternal injection or infusion, subcutaneous injection, or implant), inhalation, nasal, vaginal, rectal, sublingual, or topical routes of administration and may be formulated, alone or together, in suitable dosage unit formulations containing conventional non toxic pharmaceutically acceptable carriers, adjuvants and vehicles appropriate for each rouse of administration. The present disclosure also contemplates administration of the compounds and compositions of the present disclosure in a depot formulation.

In the treatment or prevention of conditions which require chemokine receptor modulation an appropriate dosage level will generally be about 0.001 to 100 mg per kg patient body weight per day which can be administered in single or multiple doses. Preferably, the dosage level will be about 0.01 to about 25 mg/kg per day; more preferably about 0.05 to about 10 mg/kg per day. A suitable dosage level may be about 0.01 to 25 mg/kg per day, about 0.05 to 10 mg/kg per day, or about 0.1 to 5 mg/kg per day. Within this range the dosage may be 0.005 to 0.05, 0.05 to 0.5, 0.5 to 5.0, or 5.0 to 50 mg/kg per day. For oral administration, the compositions are preferably provided in the form of tablets containing 1.0 to 1000 milligrams of the active ingredient, particularly 1.0, 5.0, 10.0, 15.0, 20.0, 25.0, 50.0, 75.0, 100.0, 150.0, 200.0, 250.0, 300.0, 400.0, 500.0, 600.0, 750.0, 800.0, 900.0, and 1000.0 milligrams of the active ingredient for the symptomatic adjustment of the dosage to the patient to be treated. The compounds may be administered on a regimen of 1 to 4 times per day, preferably once or twice per day.

It will be understood, however, that the specific dose level and frequency of dosage for any particular patient may be varied and will depend upon a variety of factors including the activity of the specific compound employed, the metabolic stability and length of action of that compound, the age, body weight, hereditary characteristics, general health, sex, diet, mode and time of administration, rate of excretion, drug combination, the severity of the particular condition, and the host undergoing therapy.

In still other embodiments, the present methods are directed to the treatment of allergic diseases, wherein a compound or composition of the disclosure is administered either alone or in combination with a second therapeutic agent, wherein said second therapeutic agent is an antihistamine. When used in combination, the practitioner can administer a combination of the compound or composition of the present disclosure and a second therapeutic agent. Also, the compound or composition and the second therapeutic agent can be administered sequentially, in any order.

The compounds and compositions of the present disclosure can be combined with other compounds and compositions having related utilities to prevent and treat the condition or disease of interest, such as inflammatory conditions and diseases, including inflammatory bowel disease, allergic diseases, psoriasis, atopic dermatitis and asthma, and those pathologies noted above. Selection of the appropriate agents for use in combination therapies can be made one of ordinary skill in the art. The combination of therapeutic agents may act synergistically to effect the treatment or prevention of the various disorders. Using this approach, one may be able to achieve therapeutic efficacy with lower dosages of each agent, thus reducing the potential for adverse side effects.

In treating, preventing, ameliorating, controlling or reducing the risk of inflammation, the compounds of the present disclosure may be used in conjunction with an anti-inflammatory or analgesic agent such as an opiate agonist, a lipoxygenase inhibitor, such as an inhibitor of 5-lipoxygenase, a cyclooxygenase inhibitor, such as a cyclooxygenase-2 inhibitor, an interleukin inhibitor, such as an interleukin-1 inhibitor, an NMDA antagonist, an inhibitor of nitric oxide or an inhibitor of the synthesis of nitric oxide, a non-steroidal anti-inflammatory agent, or a cytokine-suppressing anti-inflammatory agent, for example with a compound such as acetaminophen, aspirin, codeine, biological TNF sequestrants, fentanyl, ibuprofen, indomethacin, ketorolac, morphine, naproxen, phenacetin, piroxicam, a steroidal analgesic, sufentanyl, sunlindac, tenidap, and the like.

Similarly, the compounds of the present disclosure may be administered with a pain reliever; a potentiator such as caffeine, an H2-antagonist, simethicone, aluminum or magnesium hydroxide; a decongestant such as pseudophedrine; an antitussive such as codeine; a diuretic; a sedating or non-sedating antihistamine; a very late antigen (VLA-4) antagonist; an immunosuppressant such as cyclosporin, tacrolimus, rapamycin, EDG receptor agonists, or other FK-506 type immunosuppressants; a steroid; a non-steroidal anti-asthmatic agent such as a β2-agonist, leukotriene antagonist, or leukotriene biosynthesis inhibitor; an inhibitor of phosphodiesterase type IV (PDE-IV); a cholesterol lowering agent such as a HMG-CoA reductase inhibitor, sequestrant, or cholesterol absorption inhibitor; and an anti-diabetic agent such as insulin, α-glucosidase inhibitors or glitazones.

The weight ratio of the compound of the present disclosure to the second active ingredient may be varied and will depend upon the effective dose of each ingredient. Generally, an effective dose of each will be used. Thus, for example, when a compound of the present disclosure is combined with an NSAID the weight ratio of the compound of the present disclosure to the NSAID will generally range from about 1000:1 to about 1:1000, preferably about 200:1 to about 1:200. Combinations of a compound of the present disclosure and other active ingredients will generally also be within the aforementioned range, but in each case, an effective dose of each active ingredient should be used.

In yet another aspect, the present disclosure provides methods of treating or preventing a CCR2-mediated condition or disease by administering to a subject having such a condition or disease a therapeutically effective amount of any compound of formula (I) above. Compounds for use in the present methods include those compounds according to formula (I), those provided above as embodiments, those specifically exemplified in the Examples below, and those provided with specific structures herein. The “subject” is defined herein to include animals such as mammals, including, but not limited to, primates (e.g., humans), cows, sheep, goats, horses, dogs, cats, rabbits, rats, mice and the like. In preferred embodiments, the subject is a human.

As used herein, the phrase “therapeutically effective amount” means the amount of the subject compound that will elicit the biological or medical response of a cell, tissue, system, or animal, such as a human, that is being sought by the researcher, veterinarian, medical doctor or other treatment provider.

As used herein, the phrase “CCR2-mediated condition or disease” and related phrases and terms refer to a condition or disease characterized by inappropriate, i.e., less than or greater than normal, CCR2 functional activity. Inappropriate CCR2 functional activity might arise as the result of CCR2 expression in cells which normally do not express CCR2, increased CCR2 expression (leading to, e.g., inflammatory and immunoregulatory disorders and diseases) or decreased CCR2 expression. Inappropriate CCR2 functional activity might also arise as the result of MCP-1 secretion by cells which normally do not secrete MCP-1, increased MCP-1 expression (leading to, e.g., inflammatory and immunoregulatory disorders and diseases) or decreased MCP-1 expression. A CCR2-mediated condition or disease may be completely or partially mediated by inappropriate CCR2 functional activity. However, a CCR2-mediated condition or disease is one in which modulation of CCR2 results in some effect on the underlying condition or disease (e.g., a CCR2 antagonist results in some improvement in patient well being in at least some patients). Furthermore, MCP-2, 3 and 4 are also CCR2 ligands.

In one embodiment, the present disclosure provides a method of treating a CCR2-mediated condition or disease involving administering to a subject a safe and effective amount of the compound or composition of the disclosure.

In one embodiment, the present disclosure provides a method of treating a CCR2-mediated condition or disease involving administering to a subject a safe and effective amount of the compound or composition of the disclosure, where the CCR2-mediated condition or disease is atherosclerosis.

In one embodiment, the present disclosure provides a method of treating a CCR2-mediated condition or disease involving administering to a subject a safe and effective amount of the compound or composition of the disclosure, where the CCR2-mediated condition or disease is restenosis.

In one embodiment, the present disclosure provides a method of treating a CCR2-mediated condition or disease involving administering to a subject a safe and effective amount of the compound or composition of the disclosure, where the CCR2-mediated condition or disease is multiple sclerosis.

In one embodiment, the present disclosure provides a method of treating a CCR2-mediated condition or disease involving administering to a subject a safe and effective amount of the compound or composition of the disclosure, where the CCR2-mediated condition or disease is selected from the group consisting of inflammatory bowel disease, renal fibrosis, rheumatoid arthritis, obesity and non-insulin-dependent diabetes.

In one embodiment, the present disclosure provides a method of treating a CCR2-mediated condition or disease involving administering to a subject a safe and effective amount of the compound or composition of the disclosure, where the CCR2-mediated condition or disease is type 2 diabetes.

In one embodiment, the present disclosure provides a method of treating a CCR2-mediated condition or disease involving administering to a subject a safe and effective amount of the compound or composition of the disclosure, where the CCR2-mediated condition or disease is selected from the group consisting of chronic obstructive pulmonary disease, idiopathic pulmonary fibrosis and idiopathic pneumonia syndrome.

In one embodiment, the present disclosure provides a method of treating a CCR2-mediated condition or disease involving administering to a subject a safe and effective amount of the compound or composition of the disclosure, where the administering is oral, parenteral, rectal, transdermal, sublingual, nasal or topical.

In one embodiment, the present disclosure provides a method of treating a CCR2-mediated condition or disease involving administering to a subject a safe and effective amount of the compound or composition of the disclosure, where the compound is administered in combination with an anti-inflammatory or analgesic agent.

In one embodiment, the present disclosure provides a method of treating a CCR2-mediated condition or disease involving administering to a subject a safe and effective amount of the compound or composition of the disclosure, where an anti-inflammatory or analgesic agent is also administered.

In one embodiment, the present disclosure provides a method of modulating CCR2 function in a cell, where the CCR2 function in the cell is modulated by contacting the cell with a CCR2 modulating amount of the compound of the present disclosure.

In one embodiment, the present disclosure provides a method of treating a CCR2-mediated condition or disease involving administering to a subject a safe and effective amount of the compound or composition of the disclosure, where the disease is selected from the group consisting of pulmonary fibrosis, transplantation rejection, graft-versus-host disease and cancer.

In yet other embodiments, the present methods are directed to the treatment of psoriasis wherein a compound or composition of the disclosure is used alone or in combination with a second therapeutic agent such as a corticosteroid, a lubricant, a keratolytic agent, a vitamin D₃ derivative, PUVA and anthralin.

In other embodiments, the present methods are directed to the treatment of atopic dermatitis using a compound or composition of the disclosure either alone or in combination with a second therapeutic agent such as a lubricant and a corticosteroid.

In further embodiments, the present methods are directed to the treatment of asthma using a compound or composition of the disclosure either alone or in combination with a second therapeutic agent such as a β2-agonist and a corticosteroid.

### Preparation of CCR2 Modulators

The following examples are offered to illustrate, but not to limit, the claimed disclosure.

Additionally, those skilled in the art will recognize that the molecules claimed in this patent may be synthesized using a variety of standard organic chemistry transformations. Such methods can be carried out utilizing corresponding deuterated and optionally, other isotope-containing reagents and/or intermediates to synthesize the compounds delineated herein, or invoking standard synthetic protocols known in the art for introducing isotopic atoms to a chemical structure.

Certain general reaction types employed widely to synthesize target compounds in this disclosure are summarized in the examples. Specifically, generic procedures for sulfonamide formation, pyridine N-oxide formation and 2-aminophenyl-arylmethanone synthesis via Friedel-Crafts type approaches are given, but numerous other standard chemistries are described within and were employed routinely.

While not intended to be exhaustive, representative synthetic organic transformations which can be used to prepare compounds of the disclosure are included below.

These representative transformations include; standard functional group manipulations; reductions such as nitro to amino; oxidations of functional groups including alcohols and pyridines; aryl substitutions via IPSO or other mechanisms for the introduction of a variety of groups including nitrile, methyl and halogen; protecting group introductions and removals; Grignard formation and reaction with an electrophile; metal-mediated cross couplings including but not limited to Buckwald, Suzuki and Sonigashira reactions; halogenations and other electrophilic aromatic substitution reactions; diazonium salt formations and reactions of these species; etherifications; cyclative condensations, dehydrations, oxidations and reductions leading to heteroaryl groups; aryl metallations and transmetallations and reaction of the ensuing aryl-metal species with an electrophile such as an acid chloride or Weinreb amide; amidations; esterifications; nucleophilic substitution reactions; alkylations; acylations; sulfonamide formation; chlorosulfonylations; ester and related hydrolyses, and the like.

Certain molecules claimed in this patent can exist in different enantiomeric and diastereomeric forms and all such variants of these compounds are within the scope of the disclosure.

In the descriptions of the syntheses that follow, some precursors were obtained from commercial sources. These commercial sources include Aldrich Chemical Co., Acros Organics, Ryan Scientific Incorporated, Oakwood Products Incorporated, Lancaster Chemicals, Sigma Chemical Co., Lancaster Chemical Co., TCI-America, Alfa Aesar, Davos Chemicals, and GFS Chemicals.

Compounds of the disclosure, including those listed in the table of activities, can be made by the methods and approaches described in the following experimental section, and by the use of standard organic chemistry transformations that are well known to those skilled in the art.

Compounds of the disclosure, can be made by using procedures analogous to those used in US20060173019, U.S. Pat. No. 7,622,583, U.S. Pat. No. 8,519,135 and U.S. Pat. No. 7,884,110. Such methods can be carried out utilizing corresponding deuterated reagents or intermediates to synthesize the compounds of the disclosure. Standard synthetic protocols known in the art for introducing isotopic atoms to a chemical structure may also be used. Exemplary reagents for introducing a deuterium include D₂O and or D₂.

The specific pharmacological responses observed may vary according to and depending on the particular active compound selected or whether there are present pharmaceutical carriers, as well as the type of formulation and mode of administration employed, and such expected variations or differences in the results are contemplated in accordance with practice of the present disclosure.

Although specific embodiments of the present disclosure are herein illustrated and described in detail, the disclosure is not limited thereto. The above detailed descriptions are provided as exemplary of the present disclosure and should not be construed as constituting any limitation of the disclosure. Modifications will be obvious to those skilled in the art, and all modifications that do not depart from the spirit of the disclosure are intended to be included with the scope of the appended claims.

1. A compound of the formula (I): 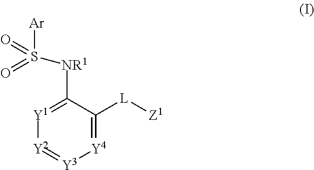
wherein
Ar is selected from the group consisting of substituted or unsubstituted optionally deuterated C₆₋₁₀ aryl and substituted or unsubstituted optionally deuterated 5- to 10-membered heteroaryl;
R¹ is selected from the group consisting of hydrogen or deuterium, substituted or unsubstituted optionally deuterated C₁₋₈ alkyl, substituted or unsubstituted optionally deuterated C₂₋₆ alkenyl, substituted or unsubstituted optionally deuterated C₂₋₆ alkynyl, and substituted or unsubstituted optionally deuterated 3- to 10-membered heterocyclyl;
Y¹ is selected from the group consisting of —CR^{2a}—, —N—, and —N⁺(O)⁻—;
Y² is selected from the group consisting of —CR^{2b}—, —N—, and —N⁺(O)⁻—;
Y³ is selected from the group consisting of —CR^{2c}—, —N—, and —N⁺(O)⁻—;
R^{2a}, R^{2b}, and R^{2c} are each independently selected from the group consisting of hydrogen or deuterium, halogen, —CN, —C(O)R³, —CO₂R³, —C(O)NR³R⁴, —OR³, —OC(O)R³, —OC(O)NR³R⁴, —SR³, —S(O)R³, —S(O)₂R³, —S(O)₂NR³R⁴, —NO₂, —NR³R⁴, —NR³C(O)R⁴, —NR³C(O)OR⁴, —NR³S(O)₂R⁴, —NR³C(O)NR⁴R⁵, substituted or unsubstituted optionally deuterated C₁₋₈ alkyl, substituted or unsubstituted optionally deuterated C₂₋₈ alkenyl, substituted or unsubstituted optionally deuterated C₂₋₈ alkynyl, substituted or unsubstituted optionally deuterated 3- to 10-membered heterocyclyl, substituted or unsubstituted optionally deuterated C₆₋₁₀ aryl, and substituted or unsubstituted optionally deuterated 5- to 10-membered heteroaryl;
R³, R⁴, and R⁵ are each independently selected from the group consisting of hydrogen or deuterium, substituted or unsubstituted optionally deuterated C₁₋₈ alkyl, substituted or unsubstituted optionally deuterated C₂₋₈ alkenyl, substituted or unsubstituted optionally deuterated C₂₋₈ alkynyl, substituted or unsubstituted optionally deuterated C₆₋₁₀ aryl, substituted or unsubstituted optionally deuterated 5- to 10-membered heteroaryl, and substituted or unsubstituted optionally deuterated 3- to 10-membered heterocyclyl;
R³ and R⁴, R⁴ and R⁵ or R³ and R⁵ may, together with the atoms to which they are attached, form a substituted or unsubstituted optionally deuterated 5-, 6-, or 7-membered ring;
L is selected from the group consisting of a bond, —O—, —S—, —S(O)—, —S(O)₂—,
—CR⁶R⁷—, —NR⁸—, —C(O)— and —NR⁸C(O)—;
R⁶ and R⁷ are each independently selected from the group consisting of hydrogen or deuterium, halogen, substituted or unsubstituted optionally deuterated C₁₋₈ alkyl, substituted or unsubstituted optionally deuterated 3- to 10-membered heterocyclyl, substituted or unsubstituted optionally deuterated C₂₋₆ alkenyl, substituted or unsubstituted optionally deuterated C₂₋₆ alkynyl, —CN, —OR⁹, —NR¹⁰R¹¹, —S(O)R⁹, and —S(O)₂R⁹;
R⁶ and R⁷ may, together with the carbon atom to which they are attached, form substituted or unsubstituted optionally deuterated C₃₋₈ cycloalkyl or substituted or unsubstituted optionally deuterated 3- to 10-membered heterocyclic ring;
R⁹ is selected from the group consisting of hydrogen or deuterium, substituted or unsubstituted optionally deuterated C₁₋₈ alkyl, substituted or unsubstituted optionally deuterated C₂₋₈ alkenyl, substituted or unsubstituted optionally deuterated C₂₋₈ alkynyl, substituted or unsubstituted optionally deuterated C₆₋₁₀ aryl, substituted or unsubstituted optionally deuterated 5- to 10-membered heteroaryl, and substituted or unsubstituted optionally deuterated 3- to 10-membered heterocyclyl;
R¹⁰ and R¹¹ are each independently selected from the group consisting of substituted or unsubstituted optionally deuterated C₁₋₈ alkyl, substituted or unsubstituted optionally deuterated 3- to 10-membered heterocyclyl, substituted or unsubstituted optionally deuterated C₆₋₁₀ aryl, substituted or unsubstituted optionally deuterated 5- to 10-membered heteroaryl, substituted or unsubstituted optionally deuterated C₂₋₈ alkenyl, and substituted or unsubstituted optionally deuterated C₂₋₈ alkynyl;
R¹⁰ and R¹¹ of —NR¹⁰R¹¹ may, together with the nitrogen, form a substituted or unsubstituted optionally deuterated C₃₋₈ cycloalkyl or substituted or unsubstituted optionally deuterated 3- to 10-membered heterocyclyl;
R⁸ is selected from the group consisting of hydrogen or deuterium,
—C(O)R¹², —S(O)₂R¹², —CO₂R¹², substituted or unsubstituted optionally deuterated C₁₋₈ alkyl, substituted or unsubstituted optionally deuterated 3- to 10-membered heterocyclyl, substituted or unsubstituted optionally deuterated C₂₋₆ alkenyl, and substituted or unsubstituted optionally deuterated C₂₋₆ alkynyl;
R¹² is selected from the group consisting of substituted or unsubstituted optionally deuterated C₁₋₈ alkyl, substituted or unsubstituted optionally deuterated C₂₋₆ alkenyl, substituted or unsubstituted optionally deuterated C₂₋₆ alkynyl, substituted or unsubstituted optionally deuterated 3- to 10-membered heterocyclyl, substituted or unsubstituted optionally deuterated C₆₋₁₀ aryl, and substituted or unsubstituted optionally deuterated 5- to 10-membered heteroaryl;
Z¹ is selected from the group consisting of substituted or unsubstituted optionally deuterated C₆₋₁₀ aryl, substituted or unsubstituted optionally deuterated 5- to 10-membered heteroaryl, substituted or unsubstituted optionally deuterated 3- to 10-membered heterocyclyl, and —NR¹³R¹⁴;
R¹³ and R¹⁴ are each independently selected from the group consisting of hydrogen or deuterium, substituted or unsubstituted optionally deuterated C₁₋₈ alkyl, substituted or unsubstituted optionally deuterated C₂₋₈ alkenyl, substituted or unsubstituted optionally deuterated C₂₋₈ alkynyl, substituted or unsubstituted optionally deuterated 3- to 10-membered heterocyclyl, substituted or unsubstituted optionally deuterated C₆₋₁₀ aryl, substituted or unsubstituted optionally deuterated 5- to 10-membered heteroaryl, substituted or unsubstituted optionally deuterated (C₁₋₄ alkyl)-(C₆₋₁₀ aryl), and substituted or unsubstituted optionally deuterated (C₁₋₄ alkyl)-(5- to 10-membered heteroaryl);
R¹³ and R¹⁴ may, together with the nitrogen, form a substituted or unsubstituted optionally deuterated 4-, 5-, 6-, or 7-membered heterocyclyl;
Y⁴ is selected from the group consisting of —N— and —N⁺(O)⁻—; and wherein the compound comprises at least one deuterium above natural isotopic abundance;
or a pharmaceutically acceptable salt thereof.

2. The compound of claim 1, wherein at least one of Y¹, Y², Y³, or Z¹ comprises deuterium above natural isotopic abundance,

3. A compound of the formula (II): 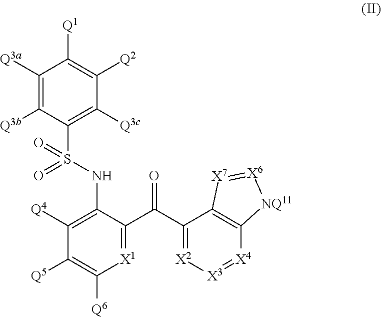
wherein
each of Q¹ and Q² independently is hydrogen, deuterium, halogen, optionally deuterated C₁₋₈ alkyl, —CN, or optionally deuterated C₁₋₈ haloalkyl;
each Q^{3a}, Q^{3b}, Q^{3c} independently is hydrogen or deuterium;
Q⁴ is hydrogen or deuterium;
Q⁵ is halogen or optionally deuterated C₁₋₈ alkyl;
Q⁶ is hydrogen or deuterium;
each of X¹, X², X⁴, X⁶, and X⁷ independently is —CQ⁷—, —N—, or —NO—;
X³ is —CQ⁷—;
each Q⁷ independently is hydrogen or deuterium, halogen, substituted or unsubstituted optionally deuterated C₁₋₈ alkyl, substituted or unsubstituted optionally deuterated C₂₋₈ alkenyl, substituted or unsubstituted optionally deuterated C₂₋₈ alkynyl, —CN, ═O, —NO₂, —OQ⁸, —OC(O)Q⁸, —CO₂Q⁸, —C(O)Q⁸, —C(O)NQ⁹Q⁸, —OC(O)NQ⁹Q⁸, —NQ¹⁰C(O)Q⁸, —NQ¹⁰C(O)NQ⁹Q⁸, —NQ⁹Q⁸, —NQ¹⁰CO₂Q⁸, —SQ⁸, —S(O)Q⁸, —S(O)₂Q⁸, —S(O)₂NQ⁹Q⁸, —NQ¹⁰S(O)₂Q⁸, substituted or unsubstituted optionally deuterated C₆₋₁₀ aryl, substituted or unsubstituted optionally deuterated 5- to 10-membered heteroaryl and substituted or unsubstituted optionally deuterated 3- to 10-membered heterocyclyl;
each occurrence of Q⁸, Q⁹, and Q¹⁰ is independently selected from the group consisting of hydrogen or deuterium, optionally deuterated C₁₋₈ alkyl, optionally deuterated C₂₋₈ alkenyl, optionally deuterated C₂₋₈ alkynyl, optionally deuterated aryl, or optionally deuterated heteroaryl; or Q⁹ and Q⁸ or Q¹⁰ and Q⁸, together with the atom(s) to which they are attached, form an substituted or unsubstituted optionally deuterated 5-, 6-, or 7-membered ring;
Q¹¹ is selected from the group consisting of hydrogen, optionally deuterated C₁₋₈ alkyl, optionally deuterated C₂₋₈ alkenyl, optionally deuterated C₂₋₈ alkynyl, optionally deuterated substituted or unsubstituted C₆₋₁₀ aryl, optionally deuterated substituted or unsubstituted 5-to 10-membered heteroaryl, and optionally deuterated substituted or unsubstituted 3- to 10-membered heterocycle; and
wherein the compound comprises at least one deuterium above natural isotopic abundance,
or a pharmaceutically acceptable salt thereof.

4. The compound of claim 3, or a pharmaceutically acceptable salt thereof, wherein at least one of Q¹ or Q² is other than hydrogen.

5. The compound of claim 3, or a pharmaceutically acceptable salt thereof, wherein each Q^{3a}, Q^{3b}, and Q^{3c} independently is hydrogen or deuterium.

6. The compound of claim 3 or a pharmaceutically acceptable salt thereof, wherein one or more of Q⁴, Q⁵, Q⁶, or Q⁷ comprises deuterium above natural isotopic abundance.

7. The compound of claim 3 or a pharmaceutically acceptable salt thereof, wherein Q¹ is halogen.

8-19. (canceled)

20. A compound selected from the group consisting of: 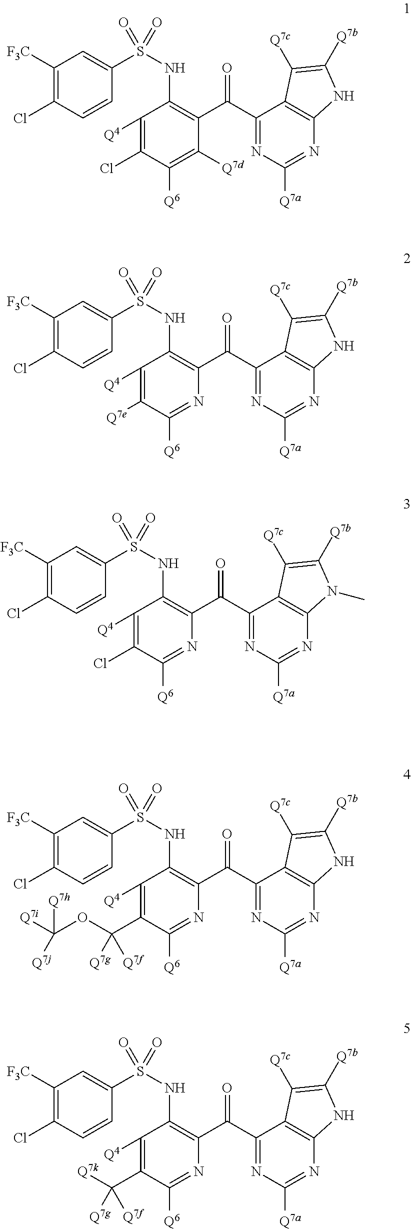 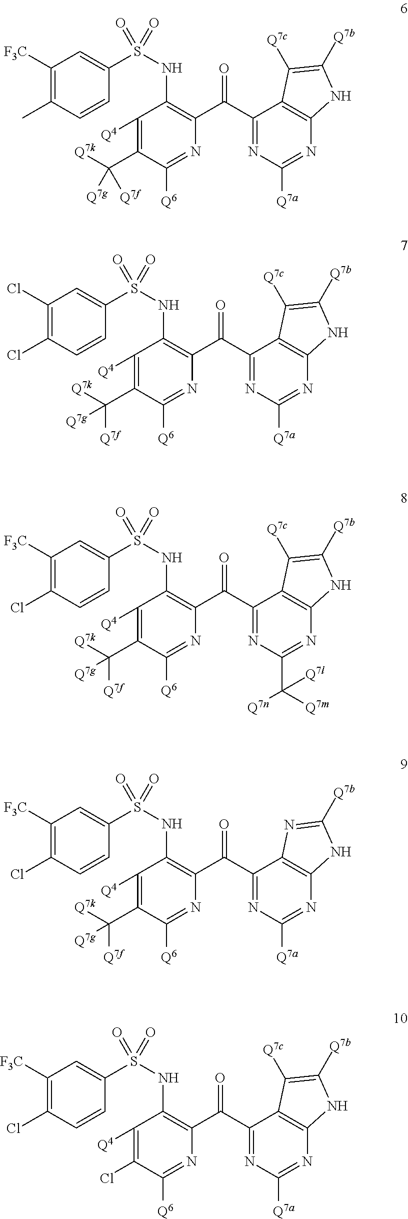 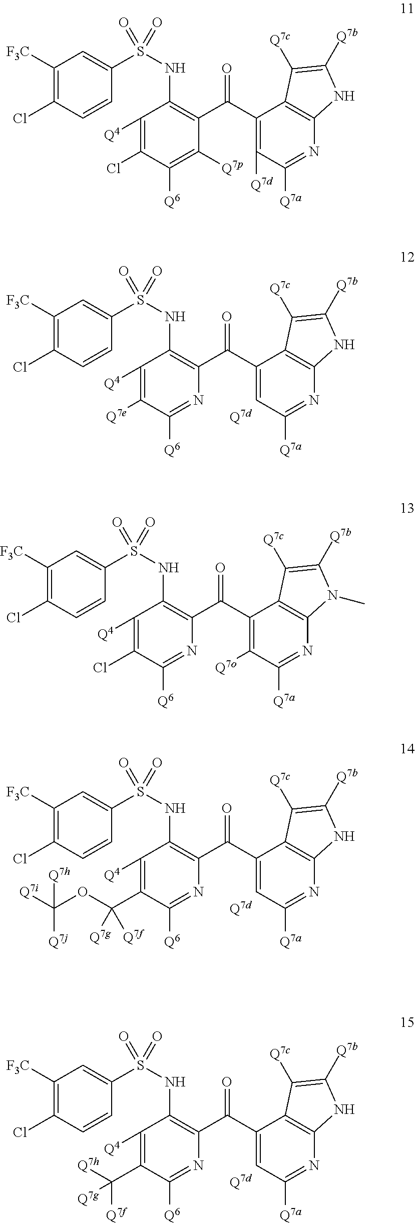 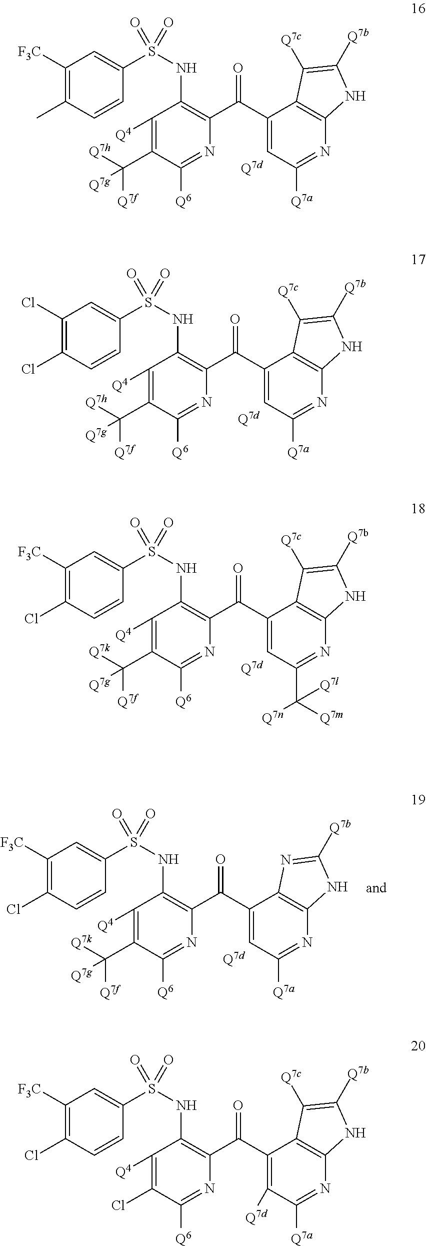
or a pharmaceutically acceptable salt thereof wherein
each Q⁴ is independently hydrogen or deuterium;
each Q⁶ is independently hydrogen or deuterium;
each Q^{7a}, Q^{7b}, Q^{7c}, Q^{7d}, Q^{7e}, Q^{7f}, Q^{7g}, Q^{7h}, Q⁷ⁱ, Q^{7j}, Q^{7k}, Q^{7l}, Q^{7m}, Q⁷ⁿ, Q^{7o}, independently is hydrogen or deuterium; and
wherein the compound comprises at least one deuterium above natural isotopic abundance.

21. The compound of claim 20, wherein, for each compound, one or more of Q⁴, Q⁶, or Q⁷ comprises deuterium above natural isotopic abundance.

22-27. (canceled)

28. A pharmaceutical composition comprising a compound of claim 1 or a pharmaceutically acceptable salt thereof and a pharmaceutically acceptable carrier.

29-30. (canceled)
